SEQUENCE LISTING (8 sequences)

SEQ ID NO: 1 - DNA, 3225 bp, Homo sapiens
  CDS (1)..(3225)
atgtcactgaaaaacgagccacgggtaaatacctctgcactgcagaaaattgctgctgacatgagtaatatcatagaaaatctggacacgcgggaactccactttgagggagaggaggtagactacgacgtgtctcccagcgatcccaagatacaagaagtgtatatccctttctctgctatttataacactcaaggatttaaggagcctaatatacagacgtatctctccggctgtccaataaaagcacaagttctggaagtggaacgcttcacatctacaacaagggtaccaagtattaatctttacactattgaattaacacatggggaatttaaatggcaagttaagaggaaattcaagcattttcaagaatttcacagagagctgctcaagtacaaagcctttatccgcatccccattcccactagaagacacacgtttaggaggcaaaacgtcagagaggagcctcgagagatgcccagtttgccccgttcatctgaaaacatgataagagaagaacaattccttggtagaagaaaacaactggaagattacttgacaaagatactaaaaatgcccatgtatagaaactatcatgccacaacagagtttcttgatataagccagctgtctttcatccatgatttgggaccaaagggcatagaaggtatgataatgaaaagatctggaggacacagaataccaggcttgaattgctgtggtcagggaagagcctgctacagatggtcaaaaagatggttaatagtgaaagattcctttttattgtatatgaaaccagacagcggtgccattgccttcgtcctgctggtagacaaagaattcaaaattaaggtggggaagaaggagacagaaacgaaatatggaatccgaattgataatctttcaaggacacttattttaaaatgcaacagctatagacatgctcggtggtggggaggggctatagaagaattcatccagaaacatggcaccaactttctcaaagatcatcgatttgggtcatatgctgctatccaagagaatgctttagctaaatggtatgttaatgccaaaggatattttgaagatgtggcaaatgcaatggaagaggcaaatgaagagatttttatcacagactggtggctgagtccagaaatcttcctgaaacgcccagtggttgagggaaatcgttggaggttggactgcattcttaaacgaaaagcacaacaaggagtgaggatcttcataatgctctacaaagaggtggaactcgctcttggcatcaatagtgaatacaccaagaggactttgatgcgtctacatcccaacataaaggtgatgagacacccggatcatgtgtcatccaccgtctatttgtgggctcaccatgagaagcttgtcatcattgaccaatcggtggcctttgtgggagggattgacctggcctatggaaggtgggacgacaatgagcacagactcacagacgtgggcagtgtgaagcgggtcacttcaggaccgtctctgggttccctcccacctgccgcaatggagtctatggaatccttaagactcaaagataaaaatgagcctgttcaaaacctacccatccagaagagtattgatgatgtggattcaaaactgaaaggaataggaaagccaagaaagttctccaaatttagtctctacaagcagctccacaggcaccacctgcacgacgcagatagcatcagcagcattgacagcacctccagttattttaatcactatagaagtcatcacaatttaatccatggtttaaaaccccacttcaaactctttcacccgtccagtgagtctgagcaaggactcactagacctcatgctgataccgggtccatccgtagtttacagacaggtgtgggagagctgcatggggaaaccagattctggcatggaaaggactactgcaatttcgtcttcaaagactgggttcaacttgataaaccttttgctgatttcattgacaggtactccacgccccggatgccctggcatgacattgcctctgcagtccacgggaaggcggctcgtgatgtggcacgtcacttcatccagcgctggaacttcacaaaaattatgaaatcaaaatatcggtccctttcttatccttttctgcttccaaagtctcaaacaacagcccatgagttgagatatcaagtgcctgggtctgtccatgctaacgtacagttgctccgctctgctgctgattggtctgctggtataaagtaccatgaagagtccatccacgccgcttacgtccatgtgatagagaacagcaggcactatatctatatcgaaaaccagtttttcataagctgtgctgatgacaaagttgtgttcaacaagataggcgatgccattgcccagaggatcctgaaagctcacagggaaaaccagaaataccgggtatatgtcgtgataccacttctgccagggttcgaaggagacatttcaaccggcggaggaaatgctctacaggcaatcatgcacttcaactacagaaccatgtgcagaggagaaaattccatccttggacagttaaaagcagagcttggtaatcagtggataaattacatatcattctgtggtcttagaacacatgcagagctcgaaggaaacctagtaactgagcttatctatgtccacagcaagttgttaattgctgatgataacactgttattattggctctgccaacataaatgaccgcagcatgctgggaaagcgtgacagtgaaatggctgtcattgtgcaagatacagagactgttccttcagtaatggatggaaaagagtaccaagctggccggtttgcccgaggacttcggctacagtgctttagggttgtccttggctatcttgatgacccaagtgaggacattcaggatccagtgagtgacaaattcttcaaggaggtgtgggtttcaacagcagctcgaaatgctacaatttatgacaaggttttccggtgccttcccaatgatgaagtacacaatttaattcagctgagagactttataaacaagcccgtattagctaaggaagatcccattcgagctgaggaggaactgaagaagatccgtggatttttggtgcaattccccttttatttcttgtctgaagaaagcctactgccttctgttgggaccaaagaggccatagtgcccatggaggtttggacttaa

SEQ ID NO: 2 - protein, 1074 aa, Homo sapiens
MSLKNEPRVNTSALQKIAADMSNIIENLDTRELHFEGEEVDYDVSPSDPKIQEVYIPFSAIYNTQGFKEPNIQTYLSGCPIKAQVLEVERFTSTTRVPSINLYTIELTHGEFKWQVKRKFKHFQEFHRELLKYKAFIRIPIPTRRHTFRRQNVREEPREMPSLPRSSENMIREEQFLGRRKQLEDYLTKILKMPMYRNYHATTEFLDISQLSFIHDLGPKGIEGMIMKRSGGHRIPGLNCCGQGRACYRWSKRWLIVKDSFLLYMKPDSGAIAFVLLVDKEFKIKVGKKETETKYGIRIDNLSRTLILKCNSYRHARWWGGAIEEFIQKHGTNFLKDHRFGSYAAIQENALAKWYVNAKGYFEDVANAMEEANEEIFITDWWLSPEIFLKRPVVEGNRWRLDCILKRKAQQGVRIFIMLYKEVELALGINSEYTKRTLMRLHPNIKVMRHPDHVSSTVYLWAHHEKLVIIDQSVAFVGGIDLAYGRWDDNEHRLTDVGSVKRVTSGPSLGSLPPAAMESMESLRLKDKNEPVQNLPIQKSIDDVDSKLKGIGKPRKFSKFSLYKQLHRHHLHDADSISSIDSTSSYFNHYRSHHNLIHGLKPHFKLFHPSSESEQGLTRPHADTGSIRSLQTGVGELHGETRFWHGKDYCNFVFKDWVQLDKPFADFIDRYSTPRMPWHDIASAVHGKAARDVARHFIQRWNFTKIMKSKYRSLSYPFLLPKSQTTAHELRYQVPGSVHANVQLLRSAADWSAGIKYHEESIHAAYVHVIENSRHYIYIENQFFISCADDKVVFNKIGDAIAQRILKAHRENQKYRVYVVIPLLPGFEGDISTGGGNALQAIMHFNYRTMCRGENSILGQLKAELGNQWINYISFCGLRTHAELEGNLVTELIYVHSKLLIADDNTVIIGSANINDRSMLGKRDSEMAVIVQDTETVPSVMDGKEYQAGRFARGLRLQCFRVVLGYLDDPSEDIQDPVSDKFFKEVWVSTAARNATIYDKVFRCLPNDEVHNLIQLRDFINKPVLAKEDPIRAEEELKKIRGFLVQFPFYFLSEESLLPSVGTKEAIVPMEVWT

SEQ ID NO: 3 - DNA, 2802 bp, Homo sapiens
  CDS (1)..(2802)
atgacggcgacccctgagagcctcttccccactggggacgaactggactccagccagctccagatggagtccgatgaggtggacaccctgaaggagggagaggacccagccgaccggatgcacccgtttctggccatctatgagcttcagtctctgaaagtgcaccccttggtgttcgcacctggggtccctgtcacagcccaggtggtgggcaccgaaagatataccagcggatccaaggtgggaacctgcactctgtattctgtccgcttgactcacggcgacttttcctggacaaccaagaagaaataccgtcattttcaggagctgcatcgggacctcctgagacacaaagtcttgatgagtctgctccctctggctcgatttgccgttgcctattctccagcccgagatgcaggcaacagagagatgccctctctaccccgggcaggtcctgagggctccaccagacatgcagccagcaaacagaaatacctggagaattacctcaactgtctcttgaccatgtctttctatcgcaactaccatgccatgacagagttcctggaagtcagtcagctgtcctttatcccggacttgggccgcaaaggactggaggggatgatccggaagcgctcaggtggccaccgtgttcctggcctcacctgctgtggccgagaccaagtttgttatcgctggtccaagaggtggctggtggtgaaggactccttcctgctgtacatgtgcctcgagacaggtgccatctcatttgttcagctctttgaccctggctttgaggtgcaagtggggaaaaggagcacggaggcacggcacggcgtgcggatcgatacctcccacaggtccttgattctcaagtgcagcagctaccggcaggcacggtggtgggcccaagagatcactgagctggcacagggcccaggcagagacttcctacagctgcaccggcatgacagctacgccccaccccggcctgggaccttggcccggtggtttgtgaatggggcaggttactttgctgctgtggcagatgccatccttcgagctcaagaggagattttcatcacagactggtggttgagtcctgaggtttacctgaagcgtccggcccattcagatgactggagactggacattatgctcaagaggaaggcggaggaaggtgtccgtgtgtctattctgctgtttaaagaagtggaattggccttgggcatcaacagtggctatagcaagagggcgctgatgctgctgcaccccaacataaaggtgatgcgtcacccagaccaagtgacgttgtgggcccatcatgagaagctcctggtggtggaccaagtggtagcattcctggggggactggaccttgcctatggccgctgggatgacctgcactaccgactgactgaccttggagactcctctgaatcagctgcctcccagcctcccaccccgcgcccagactcaccagccaccccagacctctctcacaaccaattcttctggctgggcaaggactacagcaatcttatcaccaaggactgggtgcagctggaccggcctttcgaagatttcattgacagggagacgacccctcggatgccatggcgggacgttggggtggtcgtccatggcctaccggcccgggaccttgcccggcacttcatccagcgctggaacttcaccaagaccaccaaggccaagtacaagactcccatatacccctacctgcttcccaagtctaccagcacggccaatcagctccccttcacacttccaggagggcagtgcaccaccgtacaggtcttgcgatcagtggaccgctggtcagcagggactctggagaactccatcctcaatgcctacctgcacaccatcagggagagccagcacttcctctacattgagaatcagttcttcattagctgctcagatgggcggacggttctgaacaaggtgggcgatgagattgtggacagaatcctgaaggcccacaaacaggggtggtgttaccgagtctacgtgcttttgcccttactccctggcttcgagggtgacatctccacgggcggtggcaactccatccaggccattctgcactttacttacaggaccctgtgtcgtggggagtattcaatcctgcatcgccttaaagcagccatggggacagcatggcgggactatatttccatctgcgggcttcgtacacacggagagctgggcgggcaccccgtctcggagctcatctacatccacagcaaggtgctcatcgcagatgaccggacagtcatcattggttctgcaaacatcaatgaccggagcttgctggggaagcgggacagtgagctggccgtgctgatcgaggacacagagacggaaccatccctcatgaatggggcagagtatcaggcgggcaggtttgccttgagtctgcggaagcactgcttcggtgtgattcttggagcaaatacccggccagacttggatctccgagaccccatctgtgatgacttcttccagttgtggcaagacatggctgagagcaacgccaatatctatgagcagatcttccgctgcctgccatccaatgccacgcgttccctgcggactctccgggagtacgtggccgtggagcccttggccacggtcagtccccccttggctcggtctgagctcacccaggtccagggccacctggtccacttccccctcaagttcctagaggatgagtctttgctgcccccgctgggtagcaaggagggcatgatccccctagaagtgtggacatag

SEQ ID NO: 4 - protein, 933 aa, Homo sapiens
MTATPESLFPTGDELDSSQLQMESDEVDTLKEGEDPADRMHPFLAIYELQSLKVHPLVFAPGVPVTAQVVGTERYTSGSKVGTCTLYSVRLTHGDFSWTTKKKYRHFQELHRDLLRHKVLMSLLPLARFAVAYSPARDAGNREMPSLPRAGPEGSTRHAASKQKYLENYLNCLLTMSFYRNYHAMTEFLEVSQLSFIPDLGRKGLEGMIRKRSGGHRVPGLTCCGRDQVCYRWSKRWLVVKDSFLLYMCLETGAISFVQLFDPGFEVQVGKRSTEARHGVRIDTSHRSLILKCSSYRQARWWAQEITELAQGPGRDFLQLHRHDSYAPPRPGTLARWFVNGAGYFAAVADAILRAQEEIFITDWWLSPEVYLKRPAHSDDWRLDIMLKRKAEEGVRVSILLFKEVELALGINSGYSKRALMLLHPNIKVMRHPDQVTLWAHHEKLLVVDQVVAFLGGLDLAYGRWDDLHYRLTDLGDSSESAASQPPTPRPDSPATPDLSHNQFFWLGKDYSNLITKDWVQLDRPFEDFIDRETTPRMPWRDVGVVVHGLPARDLARHFIQRWNFTKTTKAKYKTPIYPYLLPKSTSTANQLPFTLPGGQCTTVQVLRSVDRWSAGTLENSILNAYLHTIRESQHFLYIENQFFISCSDGRTVLNKVGDEIVDRILKAHKQGWCYRVYVLLPLLPGFEGDISTGGGNSIQAILHFTYRTLCRGEYSILHRLKAAMGTAWRDYISICGLRTHGELGGHPVSELIYIHSKVLIADDRTVIIGSANINDRSLLGKRDSELAVLIEDTETEPSLMNGAEYQAGRFALSLRKHCFGVILGANTRPDLDLRDPICDDFFQLWQDMAESNANIYEQIFRCLPSNATRSLRTLREYVAVEPLATVSPPLARSELTQVQGHLVHFPLKFLEDESLLPPLGSKEGMIPLEVWT

SEQ ID NO: 5 - DNA, 1473 bp, Homo sapiens
  CDS (1)..(1473)
atgaagcctaaactgatgtaccaggagctgaaggtgcctgcagaggagcccgccaatgagctgcccatgaatgagattgaggcgtggaaggctgcggaaaagaaagcccgctgggtcctgctggtcctcattctggcggttgtgggcttcggagccctgatgactcagctgtttctatgggaatacggcgacttgcatctctttgggcccaaccagcgcccagccccctgctatgacccttgcgaagcagtgctggtggaaagcattcctgagggcctggacttccccaatgcctccacggggaacccttccaccagccaggcctggctgggcctgctcgccggtgcgcacagcagcctggacatcgcctccttctactggaccctcaccaacaatgacacccacacgcaggagccctctgcccagcagggtgaggaggtcctccggcagctgcagaccctggcaccaaagggcgtgaacgtccgcatcgctgtgagcaagcccagcgggccccagccacaggcggacctgcaggctctgctgcagagcggtgcccaggtccgcatggtggacatgcagaagctgacccatggcgtcctgcataccaagttctgggtggtggaccagacccacttctacctgggcagtgccaacatggactggcgttcactgacccaggtcaaggagctgggcgtggtcatgtacaactgcagctgcctggctcgagacctgaccaagatctttgaggcctactggttcctgggccaggcaggcagctccatcccatcaacttggccccggttctatgacacccgctacaaccaagagacaccaatggagatctgcctcaatggaacccctgctctggcctacctggcgagtgcgcccccacccctgtgtccaagtggccgcactccagacctgaaggctctactcaacgtggtggacaatgcccggagtttcatctacgtcgctgtcatgaactacctgcccactctggagttctcccaccctcacaggttctggcctgccattgacgatgggctgcggcgggccacctacgagcgtggcgtcaaggtgcgcctgctcatcagctgctggggacactcggagccatccatgcgggccttcctgctctctctggctgccctgcgtgacaaccatacccactctgacatccaggtgaaactctttgtggtccccgcggatgaggcccaggctcgaatcccatatgcccgtgtcaaccacaacaagtacatggtgactgaacgcgccacctacatcggaacctccaactggtctggcaactacttcacggagacggcgggcacctcgctgctggtgacgcagaatgggaggggcggcctgcggagccagctggaggccattttcctgagggactgggactccccttacagccatgaccttgacacctcagctgacagcgtgggcaacgcctgccgcctgctctga

SEQ ID NO: 6 - protein, 490 aa, Homo sapiens
MKPKLMYQELKVPAEEPANELPMNEIEAWKAAEKKARWVLLVLILAVVGFGALMTQLFLWEYGDLHLFGPNQRPAPCYDPCEAVLVESIPEGLDFPNASTGNPSTSQAWLGLLAGAHSSLDIASFYWTLTNNDTHTQEPSAQQGEEVLRQLQTLAPKGVNVRIAVSKPSGPQPQADLQALLQSGAQVRMVDMQKLTHGVLHTKFWVVDQTHFYLGSANMDWRSLTQVKELGVVMYNCSCLARDLTKIFEAYWFLGQAGSSIPSTWPRFYDTRYNQETPMEICLNGTPALAYLASAPPPLCPSGRTPDLKALLNVVDNARSFIYVAVMNYLPTLEFSHPHRFWPAIDDGLRRATYERGVKVRLLISCWGHSEPSMRAFLLSLAALRDNHTHSDIQVKLFVVPADEAQARIPYARVNHNKYMVTERATYIGTSNWSGNYFTETAGTSLLVTQNGRGGLRSQLEAIFLRDWDSPYSHDLDTSADSVGNACRLL

SEQ ID NO: 7 - DNA, 1467 bp, Rattus rattus
  CDS (1)..(1467)
atgaagcctaaactgatgtaccaggagctgaaggttcctgttgaggaacctgcgggagaactgcccatgaatgaaatcgaggcatggaaggcagcagagaagaaagcccgttgggtcctccttgtccttatcctggcggtagtgggcttcggtgccctgatgactcagctgtttctatgggaatacggggacttacatctatttggcccgaatcagcacccagccccctgctatgacccctgcgaggcggtgctggtggagagcattcccgaggggctggagtttcccaatgccaccacaagcaacccctccaccagccaggcctggttgggcctccttgccggtgctcacagcagcctggacatcgcgtccttctactggactctcacaaacaatgatacccacacgcaagagccctctgcccagcagggtgaagaggttcttcagcagcttcaggctctggcacctcgaggtgtaaaggttcgcatcgctgtgagcaaacccaacggacctctggctgatctgcagtctctgctacagagtggtgcccaggtgcgcatggtggacatgcagaagctgacccatggtgtcctgcacaccaagttctgggtggtggaccagacccacttttacctgggcagtgccaacatggactggcgatcgctgacccaggtcaaggagctgggcgtggtcatgtacaactgcagctgcctggctcgcgacctcaccaagatttttgaagcctattggttcctgggccaggcaggcagctccatcccttcaacctggccacggccctttgacacccggtacaaccaagaaacaccgatggagatctgcctcaatggcaccccagccctggcctacctggcgagtgcacccccgccactgtgtccaggtggccgcaccccagacctgaaggcactgctcagcgtggtggacaacgcccgaagcttcatctacattgcagttatgaactacctgcccaccatggagttctcccatccacgcaggttctggccagcgattgatgatgggctaagacgggctgcgtatgaacgaggcgtcaaagtgcgtttgctcatcagctgctggggacactccgagccatccatgcggtccttcctgctctccctggctgcccttcgtgacaaccatacccactctgacatccaggtgaaactgtttgtggtccctgcggatgaggcccaagctcgaatcccctatgcccgcgtcaaccacaacaagtacatggtgactgaacgcaccacatacattggaacctccaactggtctggaagctacttcacagagacggcaggcacctccctgctggtgacacagaacgggcacggtggcttgcgcagccagctggaggctgttttcctgagagactgggaatccccatacagccacaaccttgacacctcagccgacagtgtgggcaatgcctgccgcctgctttga

SEQ ID NO: 8 - protein, 488 aa, Rattus rattus
MKPKLMYQELKVPVEEPAGELPMNEIEAWKAAEKKARWVLLVLILAVVGFGALMTQLFLWEYGDLHLFGPNQHPAPCYDPCEAVLVESIPEGLEFPNATTSNPSTSQAWLGLLAGAHSSLDIASFYWTLTNNDTHTQEPSAQQGEEVLQQLQALAPRGVKVRIAVSKPNGPLADLQSLLQSGAQVRMVDMQKLTHGVLHTKFWVVDQTHFYLGSANMDWRSLTQVKELGVVMYNCSCLARDLTKIFEAYWFLGQAGSSIPSTWPRPFDTRYNQETPMEICLNGTPALAYLASAPPPLCPGGRTPDLKALLSVVDNARSFIYIAVMNYLPTMEFSHPRRFWPAIDDGLRRAAYERGVKVRLLISCWGHSEPSMRSFLLSLAALRDNHTHSDIQVKLFVVPADEAQARIPYARVNHNKYMVTERTTYIGTSNWSGSYFTETAGTSLLVTQNGHGGLRSQLEAVFLRDWESPYSHNLDTSADSVGNACRLL

(19) United States
(12) Patent Application Publication
(10) Pub. No.: US 2007/0004657 A1
(43) Pub. Date: Jan. 4, 2007

(54) Diagnosis and treatment of kidney fibrosis and other fibrotic diseases

(76) Inventors: Orna Mor, Kirval Ono (IL); Elena Feinstein, Rehovot (IL); Hagit Shapira, Hod Hasharon (IL)

Correspondence Address: COOPER & DUNHAM, LLP, 1185 AVENUE OF THE AMERICAS, NEW YORK, NY, 10036, US

(21) Appl. No.: 10/579,662

(22) PCT Filed: Nov. 16, 2004
(86) PCT No.: PCT/IL04/01049

§ 371 Date: May 17, 2006

Related U.S. Application Data
(60) Provisional application No. 60/520,935, filed on Nov. 17, 2003.

(51) Int. Cl. A61K 48/00; A61K 39/395

(52) U.S. Cl. 514/44; 424/146.1

(57) ABSTRACT

The present invention provides methods of treatment of fibrosis by identification and isolation of polynucleotide sequences, the expression of which is altered in fibrosis. The present invention also relates to the use of these isolated polynucleotides and the polypeptides encoded thereby as probes for diagnosis, for screening of treatment modalities and as targets for modulation in kidney fibrosis and in other fibrotic conditions, including ocular scarring and cataract. In particular, the present invention provides methods, compounds and pharmaceutical compositions for the treatment of fibrosis in general, and kidney fibrosis and related pathologies and ocular scarring and cataract in particular. Novel pharmaceutical compositions comprising oligonucleotides or antibodies are also provided.

### FIELD OF THE INVENTION

The present invention relates to methods of treatment of fibrosis in general and for treatment of nephropathy, CRI, CRF, kidney fibrosis, glomerulosclerosis and ocular scarring and cataract in particular by identification and isolation of polynucleotide sequences, the expression of which is altered in fibrosis; it also relates to use of these isolated polynucleotides and the polypeptides encoded thereby as probes for diagnosis, for screening of treatment modalities and as targets for modulation in fibrosis.

### BACKGROUND OF THE INVENTION

Fibrotic Diseases

Fibrotic diseases are all characterized by the excess production of a fibrous material within the extracellular matrix, which contributes to abnormal changes in tissue architecture and interferes with normal organ function. Millions of people world-wide suffer from these chronic diseases, that are often life threatening. Unfortunately, although fibrosis is widely prevalent, debilitating and often life threatening, there is no effective treatment currently available.

The human body responds to trauma and injury by scarring. Fibrosis, a type of disorder characterized by excessive scarring, occurs when the normal wound healing response is disturbed. During fibrosis, the wound healing response continues causing excessive production and deposition of collagen.

Although fibrotic disorders can be acute or chronic, the disorders share a common characteristic of excessive collagen accumulation and an associated loss of function when normal tissue is replaced with scar tissue.

Fibrosis results from diverse causes, and may be established in various organs. Cirrhosis, pulmonary fibrosis, sarcoidosis, keloids, hypertension and kidney diseases, are all chronic diseases that induce a progressive fibrosis thereby causing a continuous loss of tissue function.

Acute fibrosis (usually with a sudden and severe onset and of short duration) occurs as a common response to various forms of trauma including accidental injuries (particularly injuries to the spine and central nervous system), infections, surgery, ischemic illness (e.g. cardiac scarring following heart attack), burns, environmental pollutants, alcohol and other types of toxins, acute respiratory distress syndrome, radiation and chemotherapy treatments. All tissues damaged by trauma are prone to scar and become fibrotic, particularly if the damage is repeated. Deep organ fibrosis is often extremely serious because the progressive loss of organ function leads to morbidity, hospitalization, dialysis, disability and even death. Fibrotic diseases or diseases in which fibrosis is evident include both acute and chronic forms like pulmonary fibrosis, interstitial lung disease, human fibrotic lung disease, liver fibrosis, cardiac fibrosis, macular degeneration, retinal and vitreal retinopathy, myocardial fibrosis, Grave's ophthalmopathy, drug induced ergotism, cardiovascular disease, atherosclerosis/restenosis, keloids and hypertrophic scars, cancer, Alzheimer's disease, scarring, scleroderma, glioblastoma in Li-Fraumeni syndrome, sporadic glioblastoma, myleoid leukemia, acute myelogenous leukemia, myelodysplastic syndrome, myeloproferative syndrome, gynecological cancer, Kaposi's sarcoma, Hansen's disease and inflammatory bowel disease, including collagenous colitis and ocular scarring and cataract.

For further information on different types of fibrosis see: Molina V, Blank M, Shoenfeld Y. (2002), “Fibrotic diseases”, Harefuah, 141(11): 973-8, 1009; Yu L, Noble N A, Border W A (2002), “Therapeutic strategies to halt renal fibrosis”, Curr Opin Pharmacol. 2(2):177-81; Keane W F, Lyle P A. (2003), “Recent advances in management of type 2 diabetes and nephropathy: lessons from the RENAAL study”, Am J Kidney Dis. 41(3 Suppl 2): S22-5; Bohle A, Kressel G, Muller C A, Muller G A. (1989), “The pathogenesis of chronic renal failure”, Pathol Res Pract. 185(4):421-40; Kikkawa R, Togawa M, Isono M, Isshiki K, Haneda M. (1997), “Mechanism of the progression of diabetic nephropathy to renal failure”, Kidney Int Suppl. 62:S39-40; Bataller R, Brenner D A. (2001), “Hepatic stellate cells as a target for the treatment of liver fibrosis”, Semin Liver Dis. 21(3):437-51; Gross T J, Hunninghake G W, (2001) “Idiopathic pulmonary fibrosis”, N Engl J Med. 345(7):517-25; Frohlich E D. (2001) “Fibrosis and ischemia: the real risks in hypertensive heart disease”, Am J Hypertens;14(6 Pt 2):194S-199S.

Liver Fibrosis

Liver fibrosis (LF) is a generally irreversible consequence of hepatic damage of several etiologies.

In the Western world, the main etiologic categories are: alcoholic liver disease (30-50%), viral hepatitis (30%), biliary disease (5-10%), primary hemochromatosis (5%), and drug-related and cryptogenic cirrhosis, unknown etiology, (10-15%). Wilson's disease, α₁-antitrypsin deficiency and other rare diseases also have liver fibrosis as one of the symptoms.

The end stage of chronic liver disease is characterized by formation of fibrous septa (scars) replacing multiple adjacent lobules, followed by parenchymal nodules created by encircled hepatocytes and eventually disruption of the architecture of the entire liver. Liver cirrhosis, the end stage of liver fibrosis, frequently requires liver transplantation and is among the top ten causes of death in the Western world.

Hepatic stellate cells (HSC) are one of the key cell types involved in the initiation and progression of liver fibrosis. In response to cytokines released by damaged hepatocytes, HSC proliferate and undergo activation and transformation from vitamin A-storing cells into collagen-producing myofibroblasts.

Anti-inflammatory agents, inhibition of activation of stellate cells, stimulation of growth of hepatocytes and inhibition of post translational modification of collagen have all been used to treat liver fibrosis. However, due to the lack of selective targeting, these treatments suffer the drawbacks of severe side effects, inter alia.

For more information on liver fibrosis see: Friedman S L. (2003), “Liver fibrosis—from bench to bedside”, J Hepatol. 38 Suppl 1:S38-53; Albanis E, Safadi R, Friedman S L. (2003), “Treatment of hepatic fibrosis: almost there”, Curr Gastroenterol Rep. 5(l):48-56.

Chronic Renal Failure (CRF)

Chronic renal failure is a gradual and progressive loss of the ability of the kidneys to excrete wastes, concentrate urine, and conserve electrolytes. CRF is slowly progressive. It most often results from any disease that causes gradual loss of kidney function and fibrosis is the main pathology that produces CRF.

CRF can range from mild dysfunction to severe kidney failure. Progression may continue to end stage renal disease (ESRD). CRF usually occurs over a number of years as the internal structures of the kidney are slowly damaged. In the early stages, there may be no symptoms. In fact, progression may be so gradual that symptoms do not occur until kidney function is less than one-tenth of normal.

Diabetic Nephropathy

Diabetic nephropathy, hallmarks of which are glomerulosclerosis and kidney fibrosis, is the single most prevalent cause of end-stage renal disease in the modern world, and diabetic patients constitute the largest population on dialysis. Such therapy is costly and far from optimal.

Transplantation offers a better outcome but suffers from a severe shortage of donors. More targeted therapies against diabetic nephropathy (as well as against other types of kidney pathologies) are not developed, since molecular mechanisms underlying these pathologies are largely unknown. Identification of an essential functional target gene that is modulated in the disease and affects the severity of the outcome of diabetes nephropathy has a high diagnostic as well as therapeutic value.

Origins of Kidney Pathology

It is known in the art that many pathological processes in the kidney eventually culminate in similar or identical morphological changes, namely glomerulosclerosis and fibrosis. Human kidney disease may evolve from various origins including glomerular nephritis, nephritis associated with systemic lupus, cancer, physical obstructions, toxins, metabolic disease and immunological diseases, all of which may cuhninate in kidney fibrosis. The meaning of this phenomenon is that different types of insults converge on the same single genetic program resulting in two hallmarks of fibrosis: the proliferation of fibroblasts and overproduction by them of various protein components of connective tissue. In addition, thickening of the basal membrane in the glomeruli accompanies interstitial fibrosis and culminates in glomerulosclerosis.

Gene Expression and Renal Disease

A useful way to assess the development of renal diseases involving fibrosis and glomerulosclerosis is to characterize gene expression in established animal models of kidney diseases. Examples of such models include inter alia: (i) fa/fa rats—animals genetically deficient in leptin receptor that develop insulin resistant diabetes (type II diabetes) with progressive diabetic nephropathy, (ii) GK rats—which are genetically manipulated, NIDDM phenotype rats, (iii) unilateral ureteral obstruction (UUO)—an animal model in which mainly kidney fibrosis is evident, without a background of diabetes. In this model the interstitial fibrosis is rapid and occurs within days following the obstruction, (iv) ⅚ nephrectomy is another useful animal model for chronic renal failure (CRF) in which fibrosis is evident.

Additional aspects of research may be based on an in vitro model system involving culture of either human or rodent fibroblasts in vitro under conditions mimicking various parameters of the cell microenvironment existing in CRF and fibrosis. These include treatment with high concentrations of glucose (modeling hyperglycemia), low concentrations of glucose, hypoxia (both modeling ischemic conditions that develop in the kidney following fibrosis and glomerulosclerosis), and TGF-β—one of the recognized pathogenic factors in fibrosis. Such in vitro model systems may complement the animal models in several important aspects: First, the system is fibroblast-specific; accordingly, none of the interferences often found in complex tissues that contain many cell types are present Second, if the cells are of human origin, they provide an insight on relevance of this research to human fibrosis. Furthermore, the insults to which the cells are exposed to are specific and of various concentrations and duration, thus enabling the investigation of both acute and chronic responses.

In conclusion, there is no effective treatment of fibrosis in general and certainly no effective treatment for kidney fibrosis and for its related pathologies, nor is there effective treatment for ocular scarring and cataract and there is a need therefore to develop novel compounds and methods of treatment for these purposes.

Phospholipase D Family

Phospholipase D enzymes (PLDs) constitute a family of phosphodiesterases that catalyze the hydrolysis of phosphatidylcholine (PtdCho) to generate choline and phosphatidic acid (PtdOH or PA), a potent lipid signaling molecule implicated in numerous physiological processes.

Since their initial characterization, PLDs from many species have been cloned and functionally important motifs have been identified. The Phospholipase D family is characterized by a conserved HXKXXXXD motif and this characteristic motif is essential for the catalytic function of PLD. Phospholipase D is involved in numerous physiological processes, including signal transduction and membrane vesicular trafficking in mammalian cells, vesicle coat recruitment, budding from the Golgi apparatus, exocytosis, endocytosis, organization of actin filaments in membrane ruffles and meiosis (Roth, M. G. (1999) Lipid regulators of membrane traffic through the Golgi complex. TRENDS Cell Biol. Vol. 9, 174-179; Liscovitch, M. et al. (2000) Phospholipase D: molecular and cell biology of a novel gene family. Biochem. J. Vol. 345, 401-415; (Pedersen et al., (1998) J Biol Chem Vol. 273(47): 31494-504). It activity, which cleaves phosphatidylcholine in response to various stimuli and thereby releasing phosphatidic acid, render its involvement in regulation of secretion, mitogenesis, or cytoskeletal changes (Steed et al. (1998) FASEB J. Vol. 12(13): 1309-17).

Its enzymatic activity, which hydrolyzes phosphatidylcholine (PtdCho) to produce phosphatidic acid (PtdOH) and choline, was originally discovered in cabbage leaves from which it was purified and cloned; these sequences were instrumental in cloning PLD from the budding yeast Saccharomyces cerevisiae (Rose, K. et al. (1995) Phospholipase D signaling is essential for meiosis. Proc. Natl. Acad. Sci. U.S.A. Vol. 92, 12151-12155). Both the plant and yeast sequences were used subsequently to identify the two major mammalian PLD enzymes—PLD1 (1074 amino acid residues) and PLD2 (933 amino acid residues) (Hammond, S. M. et al. (1995) Human ADP-ribosylation factor-activated phosphatidylcholine-specific phospholipase D defines a new and highly conserved gene family. J. Biol. Chem. Vol. 270, 29640-29643; Colley, W. C. et al. (1997) Phospholipase D2, a distinct phospholipase D isoform with novel regulatory properties that provokes cytoskeletal reorganization. Curr. Biol. Vol. 7, 191-201; Exton, J. H. (2002) Phospholipase D-structure, regulation and function. Rev. Physiol. Biochem. Pharmacol. Vol. 144, 1-94).

Mammalian PLDs have been localized to multiple organelles, including the nucleus, Golgi apparatus, lysosomes, secretory granules and plasma membrane. However, the detailed mechanisms that govern targeting of PLDs to different organelles, how their local activity is controlled or indeed the nature of PA effectors are not well understood (for recent observations on PLD localization to the Golgi apparatus and how members of this enzme family might play a role in regulating the structure of this organelle, see: Zachary F. et al. (2003), ‘Slip, sliding away’: phospholipase D and the Golgi apparatus. TRENDS Cell Biology vol. 13 no.10 540-546).

Mammalian PLD activities are subject to complex regulation by a variety of molecules, including protein kinase C (PKC) isoforms, phospholipids, and small GTPases of the ADP-ribosylation factor (ARF) Rho families, and c-src (for more details see: Exton, J. H. (2002) above; Hammond, S. M. et al. (1997). Characterization of two alternately spliced forms of phospholipase D1. Activation of the purified enzymes by phosphatidylinositol 4,5-bisphosphate, ADP-ribosylation factor, and Rho family monomeric GTP-binding proteins and protein kinase C-alpha. J. Biol. Chem. Vol. 272, 3860-3868; Powner, D. J. et al. (2002) Antigen-stimulated activation of phospholipase D1b by Rac1, ARF6, and PKCa in RBL-2H3 cells. Mol. Biol. Cell Vol. 13, 1252-1262; Han, J. M. et al. (2002) Phosphorylation-dependent regulation of phospholipase D2 by protein kinase C delta in rat pheochromocytoma PC12 cells. J. Biol. Chem. Vol. 277, 8290-8297; Brown, H. A. et al. (1993) ADP-ribosylation factor, a small GTP dependent regulatory protein, stimulates phospholipase D activity. Cell Vol. 75, 1137-1144; Cockcroft, S. et al. (1994) Phospholipase D: a downstream effector of ARF in granulocytes. Science 263, 523-526; Ktistakis, N. T. et al. (1996) Evidence that phospholipase D mediates ADP ribosylation factor-dependent formation of Golgi coated vesicles. J. Cell Biol. Vol. 134, 295-306; Chen, Y. G. et al. (1997) Phospholipase D stimulates release of nascent secretory vesicles from the trans-Golgi network. J. Cell Biol. Vol. 138, 495-504; Caumont, A. S. et al. (1998) Regulated exocytosis in chromaffin cells. Translocation of ARF6 stimulates a plasma membrane-associated phospholipase D. J. Biol. Chem. Vol. 273, 1373-1379; Kuai, J. et al. (2000) Effects of activated ADP-ribosylation factors on Golgi morphology require neither activation of phospholipase D1 nor recruitment of coatomer. J. Biol. Chem. Vol. 275, 4022-4032 3930-3942; U.S. Pat. No. 6,368,842 and WO 2000/50592).

Phospholipase D3 (PLD3), gi:23271402 is a recent addition to the phospholipase (PLD) family. It is likely to be localized to the plasma membrane through a long (50 amino acid) uncleavable signal peptide. PLD3 shows minimal sequence similarity to PLD1 (38% identity between ORFs; 16% identity between proteins (Clustal W) and PLD2 (43% identity between ORFs;17% identity between proteins (Clustal W), although the PLD active site signature is present In addition, it exhibits extensive amino acid identity with putative PLD orthologs in vaccinia virus (p37), Caeizorhabditis elegans and Dictyostelium discoideum (Pedersen K M. et al. (1998), Expression of a novel Murin Phospholipase D Homolog Coincides with Late Neuronal Development in the Forebrain. J. Biol. Chem. Vol. 273(47): 31494-31504).

p37, the major protein of the extracellular enveloped form of vaccinia virus, is involved in the biogenesis of the viral double membrane and in egress of the virus from the cell. It was found that incubation of p37 with phosphatidylcholine labeled in the fatty acyl side chains results in the production of diacylglycerol, free fatty acid, monoacylglycerol, and lysophosphatidylcholine. p37 also was found to metabolize phosphatidylethanolamine efficiently, although it has less activity toward phosphatidylinositol and minor or no activity toward phosphatidylserine. The purified enzyme also metabolized triacylglycerol to diacylglycerol but was inactive toward sn-1, 2-diacylglycerol. When expressed in insect cells, p37 also generated products expected from phospholipase C and A activities. Thus, p37 is a broad specificity lipase with phospholipase C, phospholipase A, and triacylglycerol lipase activities (Baek et al. (1997), Lipase activities of p37, the major envelope protein of vaccinia virus. J Biol Chem. Vol. 272(51): 32042-32049). Without being bound by theory, it is considered a possibility that PLD3 may have the same or similar enzymatic activity as p37, in contrast to the rest of the PLD3 family.

### SUMMARY OF THE INVENTION

The present invention identifies up- or down-regulator (responder) genes for gene therapy, diagnostics and therapeutics that have direct causal relationships between fibrosis in general, and kidney fibrosis and its related pathologies and ocular scarring and cataract, in particular More preferably, the present invention identifies the Phospholipase D genes as modulator genes.

The present invention further provides a process referred to herein as a screening assay for identifying modulators of the Phospholipase D family for treatment of kidney fibrosis and other fibrotic diseases. In particular, the present invention provides methods, compounds and pharmaceutical compositions for the treatment of kidney fibrosis and its related pathologies and ocular scarring and cataract and other fibrotic diseases by inhibition of Phospholipase D.

It is further an object of the present invention to identify and isolate novel genetic targets for development of drugs to treat fibrotic diseases such as liver fibrosis, chronic renal insufficiency and other fibrotic diseases, and usage of such targets as a tool for diagnostic and prognostic applications for fibrotic diseases.

In one embodiment, the present invention identifies up- or down-regulator (responder) genes for gene therapy, diagnostics and therapeutics that have direct causal relationships between a fibrotic disease and its related pathologies. More preferably, the present invention identifies the Phospholipase D genes as modulator genes in the above-mentioned models.

The present invention further provides a process referred to herein as a screening assay for identifying modulators, i.e., candidate or compounds or agents including but not limiting to neutralizing antibodies, peptides, peptido-mimetics, small molecules and other drugs, which bind to at least one of the Phospholipase D family of polynucleotides or polypeptides or have an effect on Phospholipase D transcription and/or expression or on Phospholipase D activity.

The compound or agent discovered by the above-mentioned screening assay that may modulate (affect) signaling via the Phospholipase D polypeptide can be used in fibrosis-related pathology to modulate collagen accumulation, fibronectin and/or MMP activity, fibroblast adhesion and migration on fibrillar collagen matrices and stellate cell and/or mesangial cell proliferation and basement membrane thickening. It can further be used to slow the pace of or inhibit glomerulosclerosis, to reduce the proliferation of fibroblasts and/or stellate cell, to inhibit the accumulation of extracellular matrix, to decrease the levels of phosphatidic acid and choline or even to inhibit their formation and to reduce or limit the formation of fibrotic regions in the target organ. It may also be used to reduce or limit the formation of fibrotic regions in other organs as described above.

### BRIEF DESCRIPTION OF THE FIGURES

FIG. 1. This figure sets forth the nucleic acid sequence of the human Phospholipase D1 gene-SEQ ID NO:1 (gi|4505872|ref|NM_{—}002662.1) ORF.

FIG. 2. This figure sets forth the amino acid sequence of the human Phospholipase D1 polypeptide-SEQ ID NO:2

FIG. 3. This figure sets forth the nucleic acid sequence of the human Phospholipase D2 gene-SEQ ID NO:3 (gi|20070140|ref|NM_{—}002663.2) ORF.

FIG. 4. This figure sets forth the amino acid sequence of the human Phospholipase D2 polypeptide-SEQ ID NO:4

FIG. 5. This figure sets forth the nucleic acid sequence of the human Phospholipase D3 gene-SEQ ID NO:5 (gi|23271402|gb|BC036327.1|) ORF.

FIG. 6. This figure sets forth the amino acid sequence of the human Phospholipase D3 polypeptide-SEQ ID NO:6.

FIG. 7. This figure sets forth the nucleic acid sequence of the rat Phospholipase D3 gene-SEQ ID NO:7 (gi|34855394|ref|XM_{—}341811.1|) ORF.

FIG. 8. This figure sets forth the amino acid sequence of the rat Phospholipase D3 polypeptide-SEQ ID NO:8.

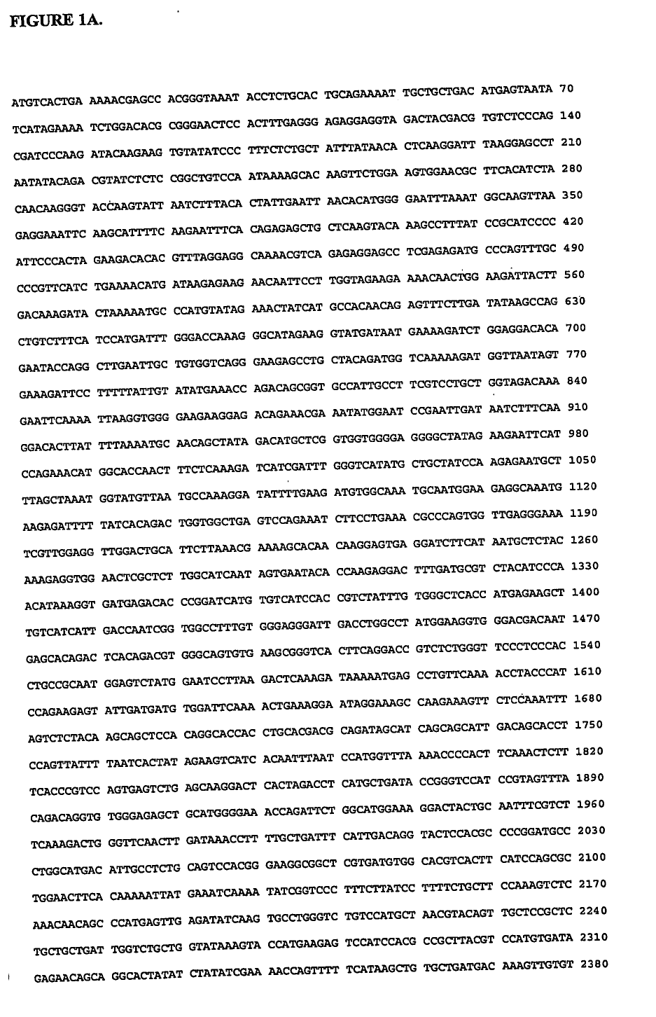

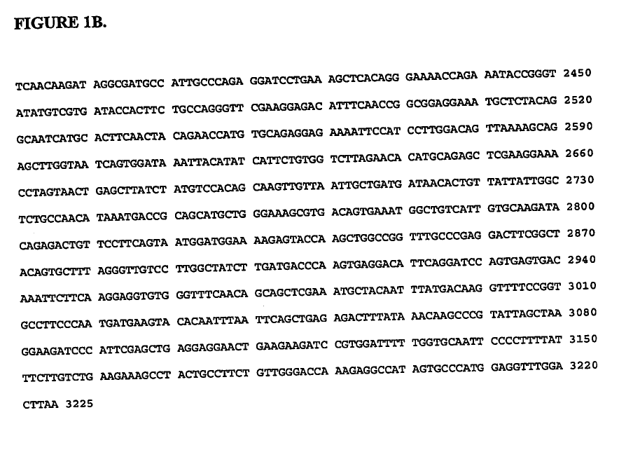

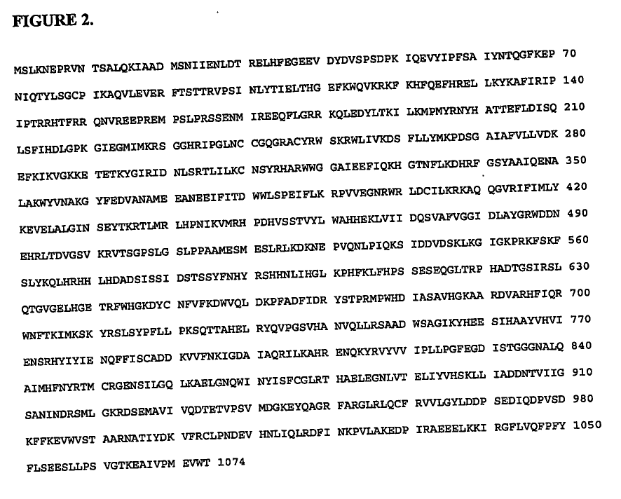

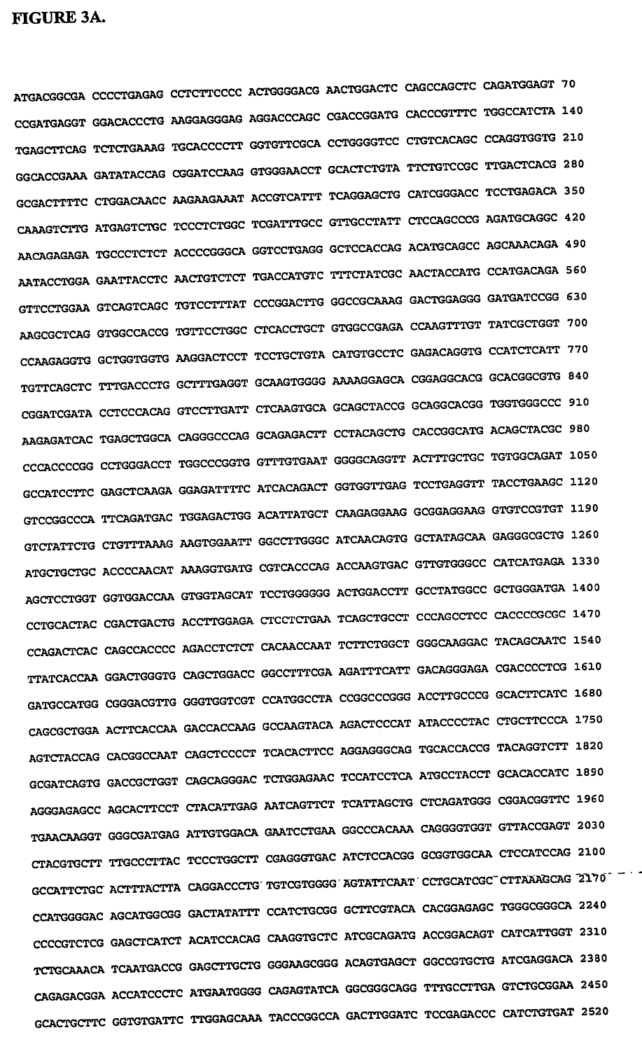

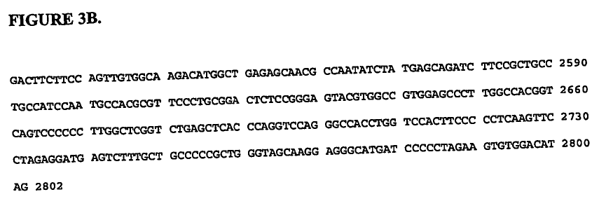

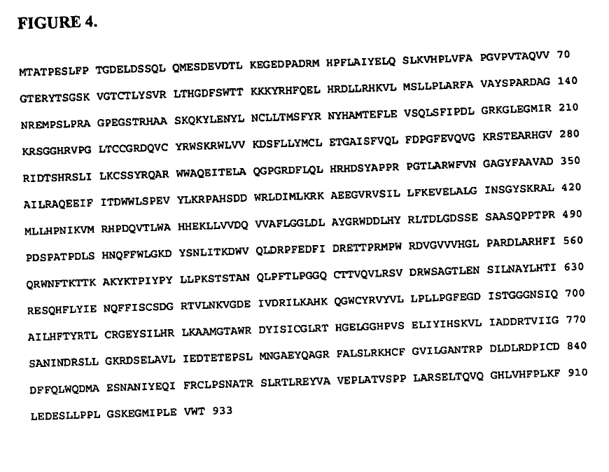

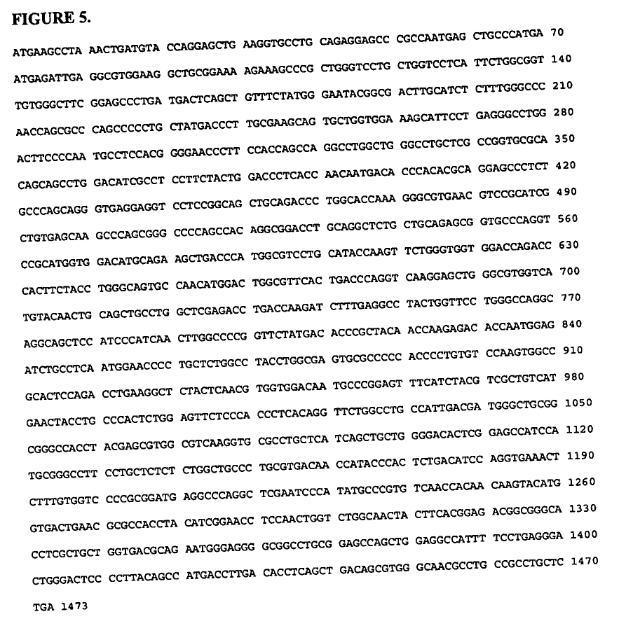

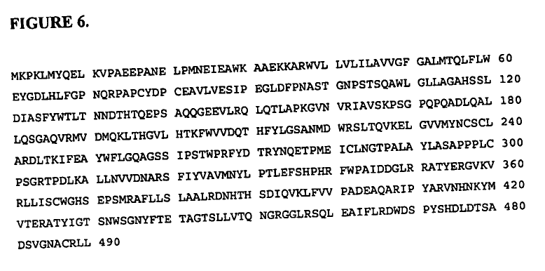

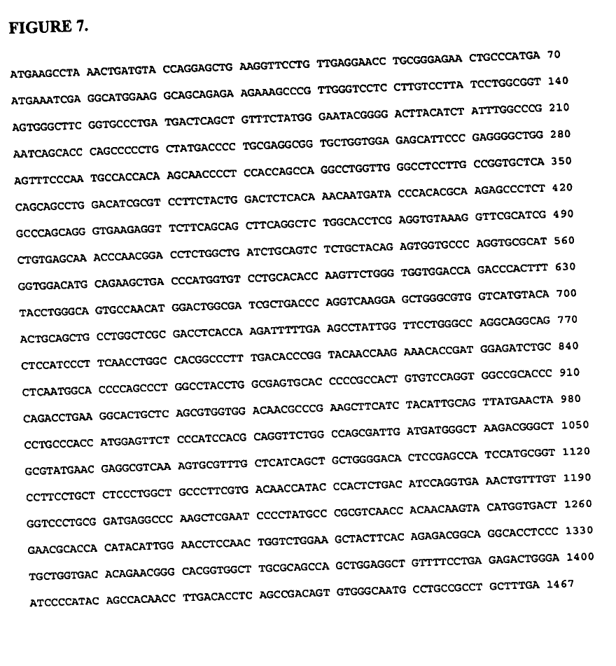

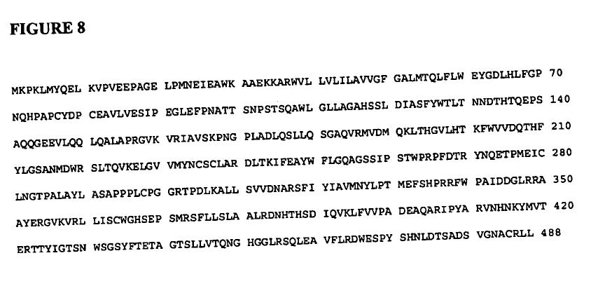

### DETAILED DESCRIPTION OF THE INVENTION

In accordance with the present invention, purified, isolated and cloned nucleic acid sequences, specifically the nucleic acid sequence that encodes the Phospholipase D polypeptide, and having sequences as specified herein or having complementary or allelic sequence variations thereto, are disclosed as being associated with fibrosis, and more specifically with fibrosis-related pathologies, and even more specifically with liver fibrosis, kidney fibrosis, pulmonary fibrosis, interstitial lung disease, human fibrotic lung disease, cardiac fibrosis, macular degeneration, chronic renal failure, diabetic nephropathy, retinal and vitreal retinopathy, myocardial fibrosis, Grave's ophthalmopathy, drug induced ergotism, cardiovascular disease, atherosclerosis, restenosis, keloids and hypertrophic scars, cancer, Alzheimer's disease, scarring, scleroderma, glioblastoma in Li-Fraumeni syndrome, sporadic glioblastoma, myleoid leukemia, acute myelogenous leukemia, myelodysplastic syndrome, myeloproferative syndrome, gynecological cancer, Kaposi's sarcoma, Hansen's disease and inflammatory bowel disease including collagenous colitis, ocular scarring and cataract. In particular, the nucleic acid sequence that encodes the Phospholipase D polypeptides have a sequence of SEQ ID NO: 1 which encodes SEQ ID NO:2 (PLD1); a sequence of SEQ ID NO:3 which encodes SEQ ID NO:4 (PLD2); or a sequence of SEQ ID NO:5 which encodes SEQ ID NO:6 (PLD3), all disclosed herein, and in particular Phospholipase D1, D2 and D3 polypeptides are deemed to be associated with fibrosis in general and with liver and kidney fibrosis in particular.

As used herein, the term “Phospholipase D gene”, or “PLD gene” is defined as the naturally occurring human gene including any allelic variant thereof as well as any homolog of either the Phospholipase D1 gene, Phospholipase D2 gene or Phospholipase D3 gene having preferably 90% homology, more preferably 95% homology, and even more preferably 98% homology to the amino acid encoding region of SEQ ID NO:1; NO:3 or No:5 correspondingly, or nucleic acid sequences which bind to the Phospholipase D1, Phospholipase D2 or Phospholipase D3 genes under conditions of highly stringent hybridization, which are well-known in the art (for example, see Ausubel et al., Current Protocols in Molecular Biology, John Wiley and Sons, Baltimore, Md. (1988), updated in 1995 and 1998).

As used herein, the term “Phospholipase D” or “PLD polypeptide” is defined as the naturally occurring human polypeptide product of any one of the Phospholipase D genes (PLD1, PLD2 or PLD3) including any allelic variant thereof as well as any homolog of either the Phospholipase D1, Phospholipase D2 or Phospholipase D3 polypeptides having preferably 90% homology, more preferably 95% homology, and even more preferably 98% homology to SEQ ID NO:2; NO:4 or No:6 correspondingly, as either full-length or fragments or a domain thereof, as a mutant of the polypeptide encoded by a spliced variant nucleic acid sequence, as a chimera with other polypeptides, provided that any of the above has the same or substantially the same biological function as the Phospholipase D polypeptides. Phospholipase D polypeptide, or a Phospholipase D polypeptide homolog, may be present in different forms, including but not limited to soluble protein, membrane-bound (either in purified membrane preparations or on a cell surface), bead-bound, or any other form presenting Phospholipase D protein or fragments and polypeptides derived thereof.

As used herein, an “interactor” is a molecule with which Phospholipase D or an Phospholipase D gene family member binds or interacts or activates in nature; for example, a molecule on the surface of a cell that expresses Phospholipase D polypeptide, a molecule on the surface of a second cell or a cytoplasmic molecule. An interactor may be a ligand that is activated by Phospholipase D alone or by Phospholipase D as part of a complex with other components. An interactor may be a component of a signal transduction pathway that facilitates transduction of an extracellular signal from Phospholipase D through the cell membrane and into the cell. An interactor, for example, can be a second intercellular protein that mediates downstream signaling from Phospholipase D.

As used herein, the term “compound” or “modulator” or “affector” or “inhibitor” is defined as comprising any small chemical molecule, antibodies, neutralizing antibodies, antisense DNA or RNA molecules, siRNA, proteins, polypeptides and peptides including peptido-mimetics and dominant negatives, and expression vectors.

In one embodiment, the invention provides assays for screening candidates or compounds or modulators or effectors that bind to, modulate the activity of, or modulate the expression level of Phospholipase D. The compounds of the present invention can be obtained by using any of the numerous approaches in combinatorial and non-combinatorial library methods known in the art, including biological libraries (proteins, peptides, etc.), spatially addressable parallel solid phase or solution phase libraries, synthetic library methods, and natural product libraries. The compound that effects the Phospholipase D expression (transcription or translation) or polypeptide activity may be inter alia a small chemical molecule which generally has a molecular weight of less than 2000 daltons, more preferably less than 1000 daltons, even more preferably less than 500 daltons. Other compounds may be antibodies preferably neutralizing antibodies or fragments thereof including single chain antibodies, antisense oligonucleotides, antisense DNA or RNA molecules, siRNA, proteins, polypeptides and peptides including peptido-mimetics and dominant negatives, and expression vectors. These compounds may act as follows: small molecules may affect expression and/or activity; antibodies—may affect activity; all kinds of antisense—may effect Phospholipase D expression; dominant negative polypeptides and peptidomimetics—may affect activity; expression vectors may be used inter alia for delivery of antisense or dominant-negative polypeptides.

Approaches have recently been developed that utilize small molecules, which can bind directly to proteins and can be used to alter protein function (for review see B. R. Stockwell, (2000) Nature Reviews/Genetics, 1, 116-125). As mentioned above, low molecular weight organic compounds can permeate the plasma membrane of target cells relatively easily and, therefore, methods have been developed for their synthesis. These syntheses, in turn, have yielded libraries that contain ligands for many proteins. Recent developments have brought a greatly increased variety of creatively selected, novel, small organic molecules that will function as powerful tools for perturbing biological systems. Such small molecules can be used to activate or inactivate specific members of a protein family.

Examples of methods for the synthesis of molecular libraries can be found in the art, for example, in DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carrell et al. (1994) Angew. Chem. Int. Ed. Engl. 33:2061; and in Gallop et al. (1994) J. Med. Chem. 37:1233.

In another aspect of the invention, Phospholipase D polypeptide can be used as “bait protein” in a two-hybrid assay or three-hybrid assay (e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO 94/10300), to identify other proteins which bind to or interact with Phospholipase D (“Phospholipase D-binding proteins”) and effect or modulate Phospholipase D activity. Such Phospholipase D-binding proteins are also likely to be involved in the propagation of signals by Phospholipase D as, for example, upstream or downstream elements of the Phospholipase D signaling pathway.

The term “treatment” as used herein refers to administration of a therapeutic substance effective to ameliorate symptoms associated with a disease, to lessen the severity or cure the disease, or to prevent the disease from occurring or from spreading.

The present invention provides for a process of obtaining a compound capable of inhibiting the activity of a human Phospholipase D polypeptide which comprises the steps of:
(i) contacting the Phospholipase D polypeptide or cells expressing Phospholipase D polypeptide with the compound;
(ii) measuring the effect of the compound on a parameter that reflects the Phospholipase D polypeptide activity in the presence of the compound; and
(iii) comparing the effect measured in step (ii) to that measured in the absence of the compound under controlled conditions, wherein a decrease in the Phospholipase D polypeptide activity identifies the compound as being capable of inhibiting the activity.

In one embodiment, the Phospholipase D polypeptide used in such process comprises consecutive amino acids, the sequence of which is set forth in SEQ ID NO: 2. In one another embodiment, the Phospholipase D polypeptide used in such process comprises consecutive amino acids, the sequence of which is set forth in SEQ ID NO:4, and in yet one another embodiment, the Phospholipase D polypeptide used in such process comprises consecutive amino acids, the sequence of which is set forth in SEQ ID NO:6. In a further embodiment the Phospholipase D3 polypeptide comprises an HXKXXXXD motif.

In another embodiment the parameter measured in any of the processes provided herein may be the presence of fibrosis, the content of collagen, fibronectin, or another extracellular matrix protein, the phosphatidic acid level or choline level, the proliferation rate of the cells or any extracellular matrix components in the cells or transdifferentiation of the cells to myofibroblasts.

In yet another embodiment, the cells used in such process are present in a tissue, and the parameter measured is, for example, the degree of inflammation in the tissue or the degree of apoptosis in the tissue.

In a further embodiment of the invention, the cells that are used for any of the processes provided are selected from hepatic cells, fibroblast cells, tubular cells and mesangial cells that express the Phospholipase D polypeptide either naturally or as a result of having been transfected with the Phospholipase D gene, either transiently or stably. In one embodiment, the fibroblast cells are renal fibroblast, the tubular cells are renal tubular cells and the mesangial cells are renal mesangial cells. In another embodiment the hepatic cells are stellate cells.

In a different embodiment, the compound in the provided processes is contacted by cells expressing Phospholipase D polypeptide. In yet a different embodiment the Phospholipase D polypeptide is contacted with the compound. The Phospholipase D polypeptide can be either immobilized or free in a solution.

In a further embodiment, the processes provided for obtaining a compound capable of inhibiting the activity of human Phospholipase D may comprise a further step in which prior to step (i) Phospholipase D is contacted with a second compound known to bind Phospholipase D.

In yet a further embodiment, the compound obtained by the provided process inhibits the activity of the Phospholipase D polypeptide at least 2-fold more effectively than it inhibits the activity of at least one other member of the Phospholipase D family, preferably both other members of the Phospholipase D family, and more preferably the inhibition is at least 10 fold, 50 fold and even 100-fold more effective.

The invention further provides a process of obtaining a compound which effects or modulates the activity of a human Phospholipase D polypeptide that comprises the steps of:
(i) contacting the Phospholipase D polypeptide with an interactor with which the Phospholipase D polypeptide interacts specifically in vivo;
(ii) contacting the Phospholipase D polypeptide or the interactor with said compound; and
(iii) measuring the effect of the compound on the interaction between Phospholipase D polypeptide and the interactor by measuring a parameter related to fibrosis; and
(iv) comparing the effect measured in step (iii) with the effect measured in the absence of the compound, a change in the effect measured indicating that the compound affects the activity of the human Phospholipase D polypeptide.

In one embodiment of the invention the compound obtained by the provided process modulates the activity of a human Phospholipase D polypeptide.

In another embodiment of the invention the Phospholipase D polypeptide in such process comprises consecutive amino acids, the sequence of which is set forth in SEQ ID NO: 2 or in SEQ ID NO:4 or in SEQ ID NO:6.

In a further embodiment of the invention the parameter measured in any of the provided processes is related to fibrosis, and more preferably to fibrosis-related pathology such as liver fibrosis, kidney fibrosis, nephropathy, cardiac fibrosis, pulmonary fibrosis, interstitial lung disease, human fibrotic lung disease, macular degeneration, retinal and vitreal retinopathy, myocardial fibrosis, Grave's ophthalmopathy, drug induced ergotism, cardiovascular disease, atherosclerosis, restenosis, keloids and hypertrophic scars, cancer, Alzheimer's disease, scarring, scleroderma, glioblastoma in Li-Fraumeni syndrome, sporadic glioblastoma, myleoid leukemia, acute myelogenous leukemia, myelodysplastic syndrome, myeloproferative syndrome, gynecological cancer, Kaposi's sarcoma, Hansen's disease and inflammatory bowel disease including collagenous colitis and ocular scarring and cataract.

In one embodiment of the invention, the compound obtained by any of the provided processes is used in a preparation of a medicament for the therapy of fibrosis-related pathology. In a different embodiment, the pathology is selected from fibrosis-related pathology as recited above. In yet a different embodiment, the pathology is selected from diabetic nephropathy, chronic renal insufficiency, chronic renal failure, kidney fibrosis and glomerulosclerosis. In yet a different embodiment, the pathology is selected from ocular scarring or cataract.

In one embodiment of the invention either the Phospholipase D polypeptide or the interactor may be immobilized.

It is an object of the present invention to provide a process of obtaining a compound capable of affecting the activity of a human Phospholipase D polypeptide that comprises the steps of:
(i) contacting the Phospholipase D polypeptide or cells expressing the Phospholipase D polypeptide with a plurality of compounds;
(ii) measuring the effect of the compounds on a parameter that reflects the Phospholipase D activity in the presence of the plurality of compounds;
(iii) comparing the effect measured in step (ii) to that measured in the absence of the plurality of compounds under controlled conditions, wherein a change identifies the plurality of compounds as being capable of affecting the activity of the human Phospholipase D polypeptide.
(iv) separately determining which compound or compounds present in the plurality effects the activity of the human Phospholipase D polypeptide.

In one embodiment of the invention, the compound obtained in such process is capable of inhibiting the activity of Phospholipase D polypeptide, and a decrease in the effect measured in steps (ii) and (iii) identifies the plurality of compounds as being capable of inhibiting the activity of Phospholipase D in either the transcription level, the translation level or the activity of the polypeptide.

It is within the scope of the present invention, to use a compound obtained according to the processes described herein, in the preparation of a medicament for treatment of fibrosis related pathology in general and for liver fibrosis, diabetic nephropathy, CRI, CRF, kidney fibrosis and glomerulosclerosis in particular and also for ocular scarring, and cataract. in particular.

Any of the screening assays according to the present invention can include a step of obtaining the compound (as described above) which tests positive in the assay, and can also include the further step of producing said compound as a medicament. It can also include steps of improving the compound to increase its desired activity before incorporating the improved compound into a medicament. It is considered that medicaments comprising such compounds are part of the present invention.

The present invention also provides a process of preparing a pharmaceutical composition which comprises:
(i) obtaining a compound that effects the activity of a human Phospholipase D polypeptide; and
(ii) admixing said compound with a pharmaceutically acceptable carrier.

In a preferred embodiment, the compound used in the preparation of a pharmaceutical composition is admixed with a carrier in a pharmaceutically effective amount

In a further embodiment, the compound on obtained by such process has an inhibition effect on Phospholipase D.

In one embodiment, this invention provides a method of treating fibrosis in general and nephropathy, CRI, CRF, kidney fibrosis and glomerulosclerosis and ocular scarring and cataract in particular in a subject which comprises administering to the subject a therapeutically effective amount of a pharmaceutical composition comprising a Phospholipase D inhibitor so as to thereby treat the subject The invention further provides such a method wherein the pharmaceutical composition comprises an oligoribonucleotide or oligonucleotide which down-regulates the expression of gene Phospholipase D by at least 50% as compared to a control. The invention further provides such a method of treating fibrosis in general and nephropathy, CRI, CRF, kidney fibrosis, glomerulosclerosis, ocular scarring and cataract in particular wherein the Phospholipase D inhibitor is an antisense oligonucleotide, a Phospholipase D siRNA, an expression vector comprising a nucleic acid molecule encoding Phospholipase D siRNA or an antibody which binds specifically to Phospholipase D polypeptide

This invention provides use of a compound which inhibits the activity of Phospholipase D in the preparation of a medicament for therapy of fibrosis in general and nephropathy, CRI, CRF, kidney fibrosis, glomerulosclerosis ocular scarring and cataract in particular. The compound which inhibits the activity of Phospholipase D is an antisense oligonucleotide, a Phospholipase D siRNA, an expression vector comprising a nucleic acid molecule encoding Phospholipase D siRNA or an antibody which binds specifically to Phospholipase D polypeptide

A pharmaceutical composition for the treatment of fibrosis in general and nephropathy, CRI, CRF, kidney fibrosis, glomerulosclerosis, ocular scarring and cataract in particular comprising as an active ingredient a Phospholipase D inhibitor together with a pharmaceutically acceptable carrier is also provided. In one embodiment the Phospholipase D inhibitor is an oligoribonucleotide or oligonucleotide which down regulates the expression of gene Phospholipase D by at least 50% as compared to a control. In other embodiments the Phospholipase D inhibitor is an antisense oligonucleotide, a Phospholipase D siRNA, an expression vector comprising a nucleic acid molecule encoding Phospholipase D siRNA or an antibody which binds specifically to Phospholipase D polypeptide.

The Phospholipase D in any of the above methods uses or compositions comprises any one of PLD1, PLD2 and PLD3.

It will be readily acknowledged by those skilled in the art that the inhibitor administered to the subject in need inhibits either the transcription or the translation or the activity of the Phospholipase D polypeptide.

The inhibitor may be, inter alia:
(a) an antisense oligonucleotide complementary to the entire or a portion of a nucleic acid molecule encoding said Phospholipase D polypeptide, said oligonucleotide being capable of inhibiting the expression of said polypeptide;
(b) a modified human Phospholipase D polypeptide which is capable of inhibiting the viability activity of the unmodified human Phospholipase D polypeptide in a dominant negative manner;
(c) a Phospholipase D siRNA;
(d) a vector capable of expressing in a cell a nucleic acid molecule encoding the antisense oligonucleotide of (a), the modified polypeptide of (b), or the siRNA of (c);
(e) an antibody capable of binding the human Phospholipase D polypeptide and partially or fully abolishing its activity; and
(f) a small chemical molecule.

Additional examples of inhibitors include ribozymes and other catalytic small RNAs, and other small oligonucleotides and polypeptides having inhibitory activity on the Phospholipase D polypeptide or on transcription/translation of a polynucleotide encoding the Phospholipase D polypeptide.

Additionally, the present invention provides a method of regulating fibrosis in a patient in need of such treatment by administering to a patient a therapeutically effective amount of at least one antisense (AS) oligonucleotide against the nucleic acid sequences or dominant negative peptide directed against the Phospholipase D sequences or Phospholipase D proteins.

In any of the provided methods the fibrosis treated in the subject may be liver fibrosis, kidney fibrosis, diabetic nephropathy, CRI, CRF or glomerulosclerosis or ocular scarring or cararact.

As used herein, “negative dominant peptide” refers to a partial cDNA sequence that encodes a part of a protein, i.e., a peptide (Herskowitz I. (1987) Nature (Review) 329(6136): 219-222). This peptide can have a function different from that of the protein from which it was derived. It can interact with a wild type protein target and inhibit its activity or it can interact with other proteins and inhibit their activity in response to the wild type target protein. Specifically, negative dominant refers to the ability of a peptide to inhibit the activity of a natural protein normally found in the cell in order to modulate the cellular phenotype, i.e., making the cell more resistant or sensitive to killing. For therapeutic intervention either the peptide itself is delivered as the active ingredient of a pharmaceutical composition or the cDNA can be delivered to the cell utilizing the same methods as for AS delivery.

The antagonist agent or regulating active ingredient is dosed and delivered in a pharmaceutically acceptable carrier as described herein below. As used herein, the term “antagonist or antagonizing” is understood in its broadest sense. Antagonism can include any mechanism or treatment that results in inhibition, inactivation, blocking or reduction in gene activity or gene product. It should be noted that the inhibition of a gene or gene product may provide for an increase in a corresponding function that the gene or gene product was regulating. The antagonizing step can include blocking cellular receptors for the gene products and can include AS, siRNA or antibody treatment as discussed below.

Many reviews have covered the main aspects of AS technology and its enormous therapeutic potential (Anazodo et al. (1995) Gene 166(2):227-232). There are reviews on the chemical aspects (Crooke S T (1995)Hematol Pathol. (Review) 9(2):59-72; Uhlmarn et al.(2000) Methods Enzymol. 313:268-284.), cellular aspects (Wagner R W (1994) Nature (Review) 372(6504):333-335), and therapeutic aspects (Hanania et al. (1995) Am J Med.(Review) 99(5):537-552; Scanlon et al. (1995) FASEB J. (Review) 9(13):1288-1296; Gewirtz A M (1993) Leuk Lymphoma. 1993;11 Suppl 1:131-137) of this rapidly developing technology. RNA interference (siRNA or RNAi) technology may also be used in the methods of this invention see, for example PCT publication WO 01/36646 (Glover et al).

By “silencing RNA” (siRNA) is meant an RNA molecule which decreases or silences the expression of a gene/mRNA of its endogenous or cellular counterpart by an RNA interference (RNAi) mechanism. For recent information on these terms and proposed mechanisms, see Bernstein E., Denli A M., Hannon G J: The rest is silence. RNA. 2001 November 7(11): 1509-21; and Nishikura K: A short primer on RNAi: RNA-directed RNA polymerase acts as a key catalyst Cell. Nov. 16, 2001;107(4):415-8.

An siRNA of the invention (Phospholipase D siRNA) normally comprises a duplex oligonucleotide in which the sense strand is derived from the mRNA sequence of Phospholipase D and the antisense strand is complementary to the sense strand. In general some deviation from the target mRNA sequence is tolerated without compromising the siRNA activity (see e.g. Czauderna et al 2003 Nucleic Acids Research 31(11),2705-2716). An siRNA of the invention inhibits gene expression on a post-transcriptional level with or without destroying the mRNA. Without being bound by theory, siRNA may target the mRNA for specific cleavage and degradation and/ or may inhibit translation from the targeted message. Each strand of the siRNA of the invention (Phospholipase D siRNA) may be about 17-40 nucleotides in length, preferably about 17 to 27 nucleotides in length, most preferably about 18 to 23 nucleotides in length for example 19 or 21 nucleotides in length. The siRNA may be modified or unmodified in its sugar residue.

siRNA compounds can be synthesized by any of the methods that are well-known in the art for synthesis of ribonucleic (or deoxyribonucleic) oligonucleotides. Such synthesis is, among others, described in Beaucage S. L. and Iyer R. P., Tetrahedron 1992; 48: 2223-2311, Beaucage S. L. and Iyer R. P., Tetrahedron 1993; 49: 6123-6194 and Caruthers M. H. et. al., Methods Enzymol. 1987; 154: 287-313, the synthesis of thioates is, among others, described in Eckstein F., Annu. Rev. Biochem. 1985; 54: 367-402, the synthesis of RNA molecules is described in Sproat 13., in Humana Press 2005 Edited by Herdewijn P.; Kap. 2: 17-31 and respective downstream processes are, among others, described in Pingoud A. et. al., in IRL Press 1989 Edited by Oliver R. W. A.; Kap. 7: 183-208 and Sproat B., in Humana Press 2005 Edited by Herdewijn P.; Kap. 2: 17-31 (supra).

Other synthetic procedures for siRNA are known in the art e.g. the procedures as described in Usman et al., 1987, J. Am. Chem. Soc., 109, 7845; Scaringe et al., 1990, Nucleic Acids Res., 18, 5433; Wincott et al., 1995, Nucleic Acids Res. 23, 2677-2684; and Wincott et al., 1997, Methods Mol. Bio., 74, 59, and these procedures may make use of common nucleic acid protecting and coupling groups, such as dimethoxytrityl at the 5′-end, and phosphoramidites at the 3′-end. The modified (e.g. 2′-O-methylated) nucleotides and unmodified nucleotides are incorporated as desired.

Strands of siRNA can be synthesized separately and joined together post-synthetically, for example, by ligation (Moore et al., 1992, Science 256, 9923; Draper et al., International PCT publication No. WO93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204), or by hybridization following synthesis and/or deprotection.

It is noted that a commercially available machine (available, inter alia, from Applied Biosystems) can be used; the siRNA oligonucleotides are prepared according to the sequences disclosed herein. Overlapping pairs of chemically synthesized fragments can be ligated using methods well known in the art (e.g., see U.S. Pat. No. 6,121,426). The strands are synthesized separately and then are annealed to each other in the tube. Then, the double-stranded siRNAs are separated from the single-stranded oligonucleotides that were not annealed (e.g. because of the excess of one of them) by HPLC. In relation to the siRNAs or siRNA fragments of the present invention, two or more such sequences can be synthesized and linked together for use in the present invention. The siRNA compounds can also be synthesized via a tandem synthesis methodology, as described in US patent application publication No. US2004/0019001(McSwiggen) wherein both siRNA strands are synthesized as a single contiguous oligonucleotide fragment or strand separated by a cleavable linker which is subsequently cleaved to provide separate siRNA fragments or strands that hybridize and permit purification of the siRNA duplex. The linker can be a polynucleotide linker or a non-nucleotide linker.

The siRNA can be delivered either directly or with viral or non-viral vectors. When delivered directly the sequences are generally rendered nuclease resistant Alternatively the sequences can be incorporated into expression cassettes or constructs such that the sequence is expressed in the cell. Generally the construct contains the proper regulatory sequence or promoter to allow the sequence to be expressed in the targeted cell. Vectors optionally used for delivery of siRNA are commercially available, and may be modified for the purpose of delivery of siRNA by methods known to one of skill in the art.

Modifications or analogs of nucleotides can be introduced to improve the therapeutic properties of the nucleotides. Improved properties include increased nuclease resistance and/or increased ability to permeate cell membranes.

Accordingly, the present invention also includes all analogs of, or modifications to, a polynucleotide or oligonucleotide of the invention that does not substantially affect the function of the polynucleotide or oligonucleotide. The nucleotides can be selected from naturally occurring or synthetically modified bases. Naturally occurring bases include adenine, guanine, cytosine, thymine and uracil. Modified bases of the oligonucleotides include xanthine, hypoxanthine, 2-aminoadenine, 6-methyl-, 2-propyl- and other alkyl-adenines, 5-halo uracil, 5-halo cytosine, 6-aza cytosine and 6-aza thymine, pseudo uracil, 4-thiuracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thioalkyl guanines, 8-hydroxyl guanine and other substituted guanines, other aza and deaza adenines, other aza and deaza guanines, 5-trifluoromethyl uracil and 5-trifluoro cytosine.

In addition, analogs of nucleotides can be prepared wherein the structures of the nucleotides are fundamentally altered and are better suited as therapeutic or experimental reagents. An example of a nucleotide analog is a peptide nucleic acid (PNA) wherein the deoxyribose (or ribose) phosphate backbone in DNA (or RNA) is replaced with a polyamide backbone similar to that found in peptides. PNA analogs have been shown to be resistant to degradation by enzymes and to have extended lives in vivo and in vitro. Further, PNAs have been shown to bind more strongly to a complementary DNA sequence than to a DNA molecule. This observation is attributed to the lack of charge repulsion between the PNA strand and the DNA strand. Other modifications that can be made to oligonucleotides include polymer backbones, cyclic backbones, or acyclic backbones. The oligonucleotide compound of the present invention consists of a multitude of nucleotides which are linked through a covalent linkage; this covalent linkage may be inter alia a phosphodiester linkage or a phosphothioate linkage, or a combination of both along the length of the nucleotide sequence of the individual strand.

The active ingredients of the pharmaceutical composition can include oligonucleotides that are nuclease resistant, needed for the practice of the invention, or a fragment thereof shown to have the same effect targeted against the appropriate sequence(s) and/or ribozymes. Combinations of active ingredients as disclosed in the present invention can be used, including combinations of AS sequences.

All oligonuclotides including AS may be synthesized and delivered by methods and vectors similar to those described for siRNA

Antibodies: By the term “antibody” as used in the present invention is meant both poly- and monoclonal complete antibodies as well as fragments thereof, such as Fab, F(ab′)2, and Fv, which are capable of binding the epitopic determinant. These antibody fragments retain the ability to selectively bind with its antigen or receptor and are exemplified as follows, inter alia:
(1) Fab, the fragment which contains a monovalent antigen-binding fragment of an antibody molecule can be produced by digestion of whole antibody with the enzyme papain to yield a light chain and a portion of the heavy chain;
(2) (Fab′)2, the fragment of the antibody that can be obtained by treating whole antibody with the enzyme pepsin without subsequent reduction; F(ab′2) is a dimer of two Fab fragments held together by two disulfide bonds;
(3) Fv, defined as a genetically engineered fragment containing the variable region of the light chain and the variable region of the heavy chain expressed as two chains; and
(4) Single chain antibody (SCA), defined as a genetically engineered molecule containing the variable region of the light chain and the variable region of the heavy chain linked by a suitable polypeptide linker as a genetically fused single chain molecule.

Such fragments having antibody functional activity can be prepared by methods known to those skilled in the art (Bird et al. (1988) Science 242:423-426)

Conveniently, antibodies may be prepared against the immunogen or portion thereof, for example, a synthetic peptide based on the sequence, or prepared recombinantly by cloning techniques or the natural gene product and/or portions thereof may be isolated and used as the immunogen. Immunogens can be used to produce antibodies by standard antibody production technology well known to those skilled in the art, as described generally in Harlow and Lane (1988), Antibodies: A Laboratory Manual, Cold Spring Harbor Laboratory, Cold Spring Harbor, N.Y., and Borrebaeck (1992), Antibody Engineering—A Practical Guide, W.H. Freeman and Co., NY.

For producing polyclonal antibodies a host, such as a rabbit or goat, is immunized with the immunogen or immunogen fragment, generally with an adjuvant and, if necessary, coupled to a carrier, antibodies to the immunogen are collected from the sera. Further, the polyclonal antibody can be absorbed such that it is monospecific; that is, the sera can be absorbed against related immunogens so that no cross-reactive antibodies remain in the sera, rendering it monospecific.

For producing monoclonal antibodies the technique involves hyperimmunization of an appropriate donor with the immunogen, generally a mouse, and isolation of splenic antibody-producing cells. These cells are fused to an immortal cell, such as a myeloma cell, to provide a fused cell hybrid that is immortal and secretes the required antibody. The cells are then cultured, in bulk, and the monoclonal antibodies harvested from the culture media for use.

For producing recombinant antibody (see generally Huston et al. (1991) “Protein engineering of single-chain Fv analogs and fusion proteins” in Methods in Enzymology (J J Langone, ed., Academic Press, New York, N.Y.) 203:46-88; Johnson and Bird (1991) “Construction of single-chain Fvb derivatives of monoclonal antibodies and their production in Escherichia coli in Methods in Enzymology (J J Langone, ed.; Academic Press, New York, N.Y.) 203:88-99; Mernaugh and Mernaugh (1995) “An overview of phage-displayed recombinant antibodies” in Molecular Methods In Plant Pathology (R P Singh and U S Singh, eds.; CRC Press Inc., Boca Raton, Fla.:359-365), messenger RNAs from antibody-producing B-lymphocytes of animals, or hybridoma are reverse-transcribed to obtain complementary DNAs (cDNAs). Antibody cDNA, which can be full or partial length, is amplified and cloned into a phage or a plasmid. The cDNA can be a partial length of heavy and light chain cDNA, separated or connected by a linker. The antibody, or antibody fragment, is expressed using a suitable expression system to obtain recombinant antibody. Antibody cDNA can also be obtained by screening pertinent expression libraries.

The antibody can be bound to a solid support substrate or conjugated with a detectable moiety or be both bound and conjugated as is well known in the art. (For a general discussion of conjugation of fluorescent or enzymatic moieties see Johnstone & Thorpe (1982.), Immunochemistry in Practice, Blackwell Scientific Publications, Oxford). The binding of antibodies to a solid support substrate is also well known in the art (for a general discussion, see Harlow & Lane (1988) Antibodies: A Laboratory Manual, Cold Spring Harbor Laboratory Publications, New York; and Borrebaeck (1992), Antibody Engineering—A Practical Guide, W.H. Freeman and Co.). The detectable moieties contemplated with the present invention can include, but are not limited to, fluorescent, metallic, enzymatic and radioactive markers such as biotin, gold, ferritin, alkaline phosphatase, β-galactosidase, peroxidase, urease, fluorescein, rhodamine, tritium, ¹⁴C and iodination.

The inhibitors of the invention may be tested for efficacy on various predictive animal models. Predictive kidney fibrosis models are described in Examples 7 and 8.

Two predictive models of liver fibrosis in rats are the Bile Duct Ligation (BDL) with sham operation as controls, and CC14 poisoning, with olive oil fed animals as controls, as described in Example 2 and in the following references:Hepatic Fibrosis: Molecular Mechanisms and Drug Targets. Lotersztajn S, Julien B, Teixeira-Clerc F, Grenard P, Mallat A Annu Rev Pharmacol Toxicol. Oct. 7, 2004; Down-regulation of connective tissue growth factor and type I collagen mRNA expression by connective tissue growth factor antisense oligonucleotide during experimental liver fibrosis. Uchio K, Graham M, Dean N M, Rosenbaum J, Desmouliere A. Wound Repair Regen. 2004 January-February;12(1):60-6. ;and Molecular classification of liver cirrhosis in a rat model by proteomics and bioinformatics. Xu X Q, Leow C K, Lu X, Zhang X, Liu J S, Wong W H, Asperger A, Deininger S, Eastwood Leung H C. Proteomics. 2004 October;4(10):3235-45.

The polypeptides of the present invention may be produced recombinantly (see generally Marshak et al., 1996 “Strategies for Protein Purification and Characterization. A laboratory course manual.” Plainview, N.Y.: Cold Spring Harbor Laboratory Press, 1996) and analogs may be produced by post-translational processing. Differences in glycosylation can provide polypeptide analogs.

As used herein, the term “polypeptide” refers to, in addition to a polypeptide, a peptide and a full protein. As used herein, “biological functional” refers to the biological property of the molecule and in this context means an in vivo effector or antigenic function or activity that is directly or indirectly performed by a naturally occurring polypeptide or nucleic acid molecule. Biological functions include but are not limited to receptor binding, any enzymatic activity or enzyme modulatory activity, any carrier binding activity, any hormonal activity, any activity in internalizing molecules or translocation from one compartment to another, any activity in promoting or inhibiting adhesion of cells to extracellular matrix or cell surface molecules, or any structural role, as well as having the nucleic acid sequence encode functional protein and be expressible. The antigenic functions essentially mean the possession of an epitope or an antigenic site that is capable of cross-reacting with antibodies raised against a naturally occurring protein. Biologically active analogs share an effector function of the native polypeptide that may, but need not, in addition possess an antigenic function. This application is also directed to a method of diagnosing a fibrosis-related pathology in a subject comprising determining in a sample from the subject the level of Phospholipase D polypeptide or the level of Phospholipase D polypeptide-encoding polynucleotide, wherein higher levels of the polypeptide or the polynucleotide compared to the levels in a subject free of such pathology is indicative of fibrosis. In a different embodiment of the invention the fibrosis may be liver fibrosis, diabetic nephropathy, CRI, CRF, kidney fibrosis or glomerulosclerosis or ocular scarring or cataract.

In a different embodiment the level of phosphatidic acid in a sample obtained from a subject in need is used as indication of fibrosis in the provided diagnostic method.

In yet another embodiment, this application provides a method of diagnosing a fibrosis-related pathology in a subject comprising contacting a body fluid sample from the subject with an antibody which specifically binds to Phospholipase D polypeptide under conditions enabling the formation of an antibody-antigen complex; and determining the level of antibody-antigen complex formed, wherein a determination of the level of antibody-antigen complex significantly higher than the level formed in a control sample indicates fibrosis in the subject

In all the diagnostic methods provided herein, the bodily fluid may be selected from the group of fluids consisting of blood, lymph fluid, ascites, serous fluid, pleural effusion, sputum, cerebrospinal fluid, lacrimal fluid, synovial fluid, saliva, stool, sperm and urine.

Measurement of the level of a Phospholipase D polypeptide i.e. PLD1, PLD2 or PLD3 may be determined by a method selected from the group consisting of immunohistochemistry, western blotting, ELISA, antibody microarray hybridization and targeted molecular imaging. Such methods are well-known in the art, for example for immunohistochemistry: M. A. Hayat (2002) Microscopy, Immunohistochemistry and Antigen Retrieval Methods: For Light and Electron Microscopy, Kluwer Academic Publishers; Brown C (1998): “Antigen retrieval methods for immunohistochemistry”, Toxicol Pathol; 26(6): 830-1); for western blotting: Laemmeli U K (1970): “Cleavage of structural proteins during the assembley of the head of a bacteriophage T4”, Nature;227: 680-685; and Egger & Bienz (1994) “Protein (western) blotting”, Mol Biotechnol; 1(3): 289-305); for ELISA: Onorato et al.(1998) “Immunohistochemical and ELISA assays for biomarkers of oxidative stress in aging and disease”, Ann NY Acad Sci 20; 854: 277-90); for antibody microarray hybridization: Huang(2001) “Detection of multiple proteins in an antibody-based protein microarray system, Immunol Methods 1; 255 (1-2): 1-13); and for targeted molecular imaging: Thomas (2001). Targeted Molecular Imaging in Oncology, Kim et al (Eds)., Springer Verlag, inter alia.

Measurement of level of Phospholipase D polynucleotide may be determined by a method selected from: RT-PCR analysis, in-situ hybridization, polynucleotide microarray and Northern blotting. Such methods are well-known in the art, for example for in-situ hybridization Andreeff & Pinkel (Editors) (1999), “Introduction to Fluorescence In Situ Hybridization: Principles and Clinical Applications”, John Wiley & Sons Inc.; and for Northern blotting Trayhurn (1996) “Northern blotting”, Proc Nutr Soc; 55(1B): 583-9 and Shifinan & Stein (1995) “A reliable and sensitive method for non-radioactive Northern blot analysis of nerve growth factor mRNA from brain tissues”, Journal of Neuroscience Methods; 59: 205-208 inter alia.

Measurement of effect of the compound on a parameter related to kidney fibrosis and comparing the effect measured with the effect measured in the absence of the compound may be determined by any of the methods described in the examples of the present invention or by any method known to a men skilled in the art.

In another embodiment of the invention, a method for the treatment of fibrosis-related pathology in a subject in need of such treatment comprising administering to said subject an amount of a modulator of Phospholipase D polypeptide sufficient to effect a substantial modulation of the Phospholipase D activity so as to thereby treat the subject, is provided. In another embodiment the modulator is an antibody, and the fibrosis-related pathology is either liver fibrosis, nephropathy, CRI, CRF, kidney fibrosis or glomerulosclerosis.

### Methods

General Methods in Molecular Biology

Standard molecular biology techniques known in the art and not specifically described were generally followed as in Sambrook et al., Molecular Cloning: A Laboratory Manual, Cold Spring Harbor Laboratory Press, New York (1989), and as in Ausubel et al., Current Protocols in Molecular Biology, John Wiley and Sons, Baltimore, Md. (1989) and as in Perbal, A Practical Guide to Molecular Cloning, John Wiley & Sons, New York (1988), and as in Watson et al., Recombinant DNA, Scientific American Books, New York and in Birren et al (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) was carried out generally as in PCR Protocols: A Guide To Methods And Applications, Academic Press, San Diego, Calif. (1990). In situ (In cell) PCR in combination with Flow Cytometry can be used for detection of cells containing specific DNA and mRNA sequences (Testoni et al., 1996, Blood 87:3822.)

General Methods in Immunology

Standard methods in immunology known in the art and not specifically described are generally followed as in Stites et al (eds), Basic and Clinical Immunology (8th Edition), Appleton & Lange, Norwalk, Conn. (1994) and Mishell and Shiigi (eds), Selected Methods in Cellular Immunology, W.H. Freeman and Co., New York (1980).

Immunoassays

In general ELISAs, where appropriate, are one type of immunoassay employed to assess a specimen. ELISA assays are well known to those skilled in the art. Both polyclonal and monoclonal antibodies can be used in the assays. Where appropriate other immunoassays, such as radioimmunoassays (RIA) can be used as are known to those skilled in the art. Available immunoassays are extensively described in the patent and scientific literature. See, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521 as well as Sambrook et al., Molecular Cloning: A Laboratory Manual, Cold Springs Harbor, N.Y., 1989.

Recombinant Protein Purification

For standard purification, See Marshak et al. (1996), “Strategies for Protein Purification and Characterization. A laboratory course manual.” CSHL Press.

Transgenic and Knockout Methods

The present invention provides for a transgenic gene and a polymorphic gene animal and cellular (cell line) model, as well as for a knockout model. These models are constructed using standard methods known in the art and as set forth in U.S. Pat. Nos. 5,487,992; 5,464,764; 5,387,742; 5,360,735; 5,347,075; 5,298,422; 5,288,846; 5,221,778; 5,175,385; 5,175,384; 5,175,383; 4,736,866; as well as Burke and Olson (1991) “Preparation of Clone Libraries in Yeast Artificial-Chromosome Vectors” in Methods in Enzymology, 194, “Guide to Yeast Genetics and Molecular Biology”, eds. C. Guthrie and G. Fink, Academic Press, Inc., Chap. 17:251-270; Capecchi (1989) “Altering the genome by homologous recombination”, Science, 244:1288-1292; Davies et al. (1992) “Targeted alterations in yeast artificial chromosomes for inter-species gene transfer”, Nucleic Acids Research, 20 (11): 2693-2698; Dickinson et al. (1993) “High frequency gene targeting using insertional vectors”, Human Molecular Genetics, 2(8):1299-1302; Duff and Lincoln (1995) “Insertion of a pathogenic mutation into a yeast artificial chromosome containing the human APP gene and expression in ES cells”, Research Advances in Alzheimer's Disease and Related Disorders Khalid Iqbal (Editor), James A. Mortimer (Editor), Bengt Winblad (Editor), Henry M. Wisniewski (Editor); Huxley et al. (1991) “The human HPRT gene on a yeast artificial chromosome is functional when transferred to mouse cells by cell fusion”, Genomics, 9:742-750; Jakobovits et al. (1993) “Germ-line transmission and expression of a human-derived yeast artificial chromosome”, Nature, 362: 255-261; Lamb et al. (1993) “Introduction and expression of the 400 kilobase precursor amyloid protein gene in transgenic mice”, Nature Genetics, 5:22-29; Pearson and Choi (1993) Expression of the human b-amyloid precursor protein gene from a yeast artificial chromosome in transgenic mice. Proc. Natl. Acad. Sci. (USA), 90:10578-10582; Rothstein, (1991) “Targeting, disruption, replacement, and allele rescue: integrative DNA transformation in yeast” in Methods in Enzymology, 194, “Guide to Yeast Genetics and Molecular Biology”, eds. C. Guthrie and G. Fink, Academic Press, Inc., NY, Chap. 19:281-301; Schedl et al. (1993) “A yeast artificial chromosome covering the tyrosinase gene confers copy number-dependent expression in transgenic mice”, Nature, 362:258-261; Strauss et al. (1993) “Germ line transmission of a yeast artificial chromosome spanning the murine al (1) collagen locus”, Science, 259:1904-1907. Further, PCT patent applications WO 94/23049, WO 93/14200, WO 94/06908, WO 94/28123 also provide information.

Further one parent strain, instead of carrying a direct human transgene, may have the homologous endogenous gene modified by gene targeting such that it approximates the transgene. That is, the endogenous gene has been “humanized” and/or mutated (Reaume et al. (1996) J Biol Chem. 271(38):23380-23388.). It should be noted that if the animal and human sequences are essentially homologous, a “humanized” gene is not required. The transgenic parent can also carry an overexpressed sequence, either the non-mutant or a mutant sequence and humanized or not as required. Herein, the term “tansgene” is therefore used to refer to all these possibilities.

Additionally, cells can be isolated from the offspring that carry a transgene from each transgenic parent and that are used to establish primary cell cultures or cell lines as is known in the art.

Where appropriate, a parent strain will be homozygous for the transgene. Additionally, where appropriate, the endogenous non-transgene in the genome that is homologous to the transgene will be non-expressive. Herein, by the term “non-expressive” is meant that the endogenous gene will not be expressed and that this non-expression is heritable in the offspring. For example, the endogenous homologous gene could be “knocked-out” by methods known in the art. Alternatively, the parental strain that receives one of the transgenes could carry a mutation at the endogenous homologous gene rendering it non-expressed.

The present invention is illustrated in detail below with reference to Examples, but is not to be construed as being limited thereto.

### EXAMPLES

### Example 1

### Preparation of Dedicated Rat Liver Fibrosis cDNA Microarray Array (RLF)

Three cDNA libraries were prepared in accordance with the proprietary methodology of the assignee, essentially as described in co-assigned U.S. Pat. No. 6,544,741. These three libraries are:
a) RSR library, derived from cultured hepatic stellate cells, either untreated or treated with TGFβ, or PDGF or a combination of both compounds;
b) RSV library, derived from freshly isolated hepatic stellate cells from CCl₄ and Bile Duct Ligation (BDL) treated animals;
c) RLV library, derived from total liver tissue samples excised from the same CCl₄ and BDL treated animals.

3000 cDNA clones were partially sequenced and annotated during the preparation of these libraries, yielding 1322 different non-redundant clones.

The proportion of each library to be printed on the array was determined on the basis of the analysis of the annotated clones, according to the following criteria:
a. Percent of redundancy in each library.
b. Similarity of clone representation between different libraries. (The analysis revealed high similarity between the RSV and RLV libraries.)

Accordingly, the following clones were printed on the array:
1728 pre-annotated clones;
3456 clones from the RSR library;
3744 clones from the RSV library;
1056 clones from the RLV library.

A total of 9984 clones were printed on the RLF array.

### Example 2

### In vivo Experiments for Preparation of Hybridization Probes

Two models of liver fibrosis in rats were employed, the Bile Duct Ligation (BDL) with sham operation as controls, and CC14 poisoning, with olive oil fed animals as controls. Partial Hepatectomy (PH) was utilized as a model for liver regeneration. Three types of samples were obtained for each of these models:
1. Frozen isolated hepatic stellate cells
2. Total liver tissue

(Samples 1 and 2 were used for production of RNA probes)

3. Formalin-fixed liver tissues, which were used to evaluate the extent of fibrosis in each animal and for in situ hybridization analysis of pre candidate genes expression.

TABLE 1
BDL and PH experimental platform.

| Liver fibrosis and | | | | | | | |
|---|---|---|---|---|---|---|---|
| Regeneration models | 0′ | 6 h | 24 h | 48 h | 72 h | 4 w | 5 w |
| Untreated Control | o, o | | | | | | |
| Common bile duct | | o | o | o | o | o | o |
| ligation | | | | | | | |
| Sham (Control for | | o | o | o | o | o | o |
| bile duct ligation) | | | | | | | |
| Partial Hepatectomy | | | o | o | o | | |
| (regeneration) | | | | | | | |

o - sampling

TABLE 2
CCl₄ model olive oil as control - experimental platform.

| Time | Number of treatments | | | | | | | |
|---|---|---|---|---|---|---|---|---|
| harvest | none | 1^{st} | 2^{nd} | 3^{rd} | 4^{th} | 5^{th} | 6^{th} | 12^{th} |
| O | o | + | | | | | | |
| Day 1 | | o | | | | | | |
| Day 2 | | o | | | | | | |
| Day 3 | | o | + | | | | | |
| Day 6 | | | o | + | | | | |
| Day 9 | | | | o | + | | | |
| Day 12 | | | | o | o | + | | |
| Day 15 | | | | o | | o | + | |
| Day 16 | | | | | | | o | |
| Day 17 | | | | | | | o | |
| Day 18 | | | | | | | o | |
| Day 21 | | | | | | | o | |
| Day 24 | | | | | | | o | |
| Day 33 | | | | | | | | + |
| Day 36 | | | | | | | | o |
| Day 39 | | | | | | | | o |
| Day 42 | | | | | | | | o |
| Day 45 | | | | | | | | o |
| Day 48 | | | | | | | | o |

+ - treatment;
o - sampling

### Example 3

### Histological Evaluation of Fibrosis in Liver Samples

1. Histological Evaluation of Fibrosis Status of Formalin-Fixed Liver Samples

Formalin fixed liver samples were embedded into paraffin and 5 μm sections were prepared. Each sample was analyzed by 5 independent methods:
1. Hematoxylin/Eosin (HE) histochemical staining for evaluation of morphological changes.
2. Sirius red histochemical staining for collagen accumulation.
3. Inmunohistochemical staining with anti-α-smooth muscle actin antibodies for evaluation of localization and number of activated fibrotic cells.
4. In situ hybridization with a TGFβ-specific probe and with collagen type I-specific probe for evaluation of their expression level.

A total of 260 samples were evaluated by all 5 methods. Out of these samples, 12 samples were disqualified for probe preparation due to exceptional fibrotic performance (either exceptionally more or less severe than the average picture in the same group).

2. Histological Analysis of CCl₄ Induced Hepatic Damage

1-6 repeated CCl₄ treatments usually result in acute damage to liver parenchyma. Examination of HE-stained samples obtained 1 and 3 days after each treatment revealed acute centrilobular necrosis with inflammatory infiltration, without marked reaction in portal fields. It was found that at least 3 consequent CCl₄ treatments should be applied in order to induce (as observed on day 3 after the last treatment) a prominent accumulation of collagen (Sirius Red staining) and αSMA-positive myofibroblasts in immature septa, developing within pericentral necroinflammatory zones. Cessation of CCL₄ poisoning after 3 treatments resulted in recovery of acute damage and regression of fibrotic changes within 9 days characterized according to the following:
Spreading of pericentral postnecrotic zones was markedly reduced, albeit fat transformation of hepatocytes in pericentral zones persisted;
Sirius Red staining demonstrated significant regression of fibrotic septa;
αSMA-positive myofibroblasts almost disappeared.
Level of TGFβ1 and Coll1 expression was much lower than at the peak of acute response, but still higher than in normal liver.

The continuation of CCl₄ treatment resulted in more sustained liver damage. As observed at day 3 following six CCl₄ treatments, branching fibrotic septa evolved within strip-like necroinflammatory fields in parenchyma. These fields tend to separate parenchyma, forming central-central and sometimes central-portal bridging patterns. Apoptotic bodies and mitotic hepatocytes were evident in parenchyma. Mild bile duct proliferation and slight portal fibrosis were also noted. On day 3 after 12 treatments the liver samples displayed complete destruction of lobular architecture. Separation of parenchyma by rough, fibrous septa, regenerative patterns within separated parenchymal nodules, and portal fibrosis with extensive proliferation of bile ducts were observed. These findings are indicative of advanced fibrosis and nodular cirrhosis. On day 15 after the cessation of the treatment some reduction in the density of fibrotic septa was observed, but regenerative nodules were still present.

Sirius Red staining in samples with maximal fibrotic development outlined fibrillary branching structures within necroinflammatory fields, suggesting significant collagen precipitation within septa. Immunostaining for αSMA with these samples revealed a branching network formed by numerous αSMA-positive myofibroblasts within necroinflammatory fields. Single myofibroblasts were scattered in parenchyma and in portal fields. Strong up-regulation of TGFβ1 expression was observed within 24 hr following each treatment and remained high up to day 3. It was found that activation of Collagen I expression lags behind TGFβ1. Expression of both TGFβ1 and Collagen I was stronger in repeatedly treated samples compared to those derived from rats treated with a single dose, though the pattern of acute reactivity to CCl₄ administration was evident regardless of the number of treatments that the animal obtained.

In summary, the CCl₄ model as designed provides an insight into three distinct phases of fibrogenesis: acute (transient), chronic (persistent) and recovery phases:
a) 1-4 treatments cause acute centrilobular necrosis, resulting in mild and reversible fibrotic changes in parenchyma.
b) 5-6 treatments lead to more pronounced centrilobular necrotic and fibrotic changes, and initial signs of portal fibrosis. These changes are reversible to a certain extent and do not lead to development of cirrhosis.
c) 12 treatments result in severe fibrotic changes in parenchyma and portal tracts and cause development of cirrhosis. Cessation of poisoning does not lead to significant reversal of fibrotic and cirrhotic changes (at least within 15 days).

3. Histological Analysis of Common Bile Duct Ligation (BDL) Samples

BDL for four weeks caused expansion of portal fields, containing multiple proliferating bile ducts and entrapped islets of degenerating hepatocytes. Severe portal fibrosis was evident by Sirius red staining of collagen deposits along portal tracts and around proliferating bile ducts. Centrilobular architecture with preserved central veins and pericentral hepatocytes was present in part of the lobules, however excessive deposition of Sirius red stained fibrous material around central veins and signs of capillarization of sinuses were recorded.

TGFβ1 was diffusely expressed in parenchyma. Stronger expression was observed around some of the proliferating bile ducts, and in inflammatory cells that infiltrate nodules of degenerating parenchyma.

Coll1 was moderately elevated around proliferating bile ducts. Stronger expression was also observed within fibrotic septa and in nodules of degenerating parenchyma.

BDL for five weeks caused further expansion of portal fields resulting in more massive entrapping of hepatocytes and in portal-portal bridging. Sirius Red staining revealed significant elevation of collagen deposition in portal fields in comparison to the four week specimens.

The destructive changes in centrilobular architecture also became more prominent: Remaining central veins were bridged by outgrowing portal extensions and Sirius red staining revealed collagen fibres precipitating in the parenchyma.

Regenerative zones as well as scarred postnecrotic areas were observed in parenchyma. Both in four week and five week specimens an extensive network of αSMA-positive myofibroblasts surrounded proliferating bile ducts. Myofibroblasts were observed also within scarred areas and free in parenchyma.

Thus, four weeks of BDL provided a time point for advanced portal fibrosis. During the following week clear transition of this destructive process into the cirrhosis stage took place.

In summary, the BDL model enables the detection of genes involved in the chronic progression of the disease.

### Example 4

### RLF Hybridizations Results

Total RNA was prepared from pellets of frozen hepatic stellate cells using EASY™ KIT (Biological Industries, Israel), according to the manufacturer's instructions. The RNA samples corresponding to each type of treatment were then pooled for PolyA⁺ RNA preparation and labeled with Cy5. PolyA⁺ RNA isolated from hepatic stellate cells of control (untreated) animals was with Cy3 and served as common control for all hybridizations.

The list of probes for this set of hybridization is summarized below in tables 3 and 4.

TABLE 3
Probes derived from the BDL model.

| Probe number | Treatment | Time after treatment | |
|---|---|---|---|
| 1 | BDL | 6 | h |
| 2 | BDL | 24 | h |
| 3 | BDL | 48 | h |
| 4 | BDL | 72 | h |
| 38 | BDL | 4 | w |
| 39 | BDL | 5 | w |
| 5 | Sham | 6 | h |
| 6 | Sham | 24 | h |
| 7 | Sham | 48 | h |
| 8 | Sham | 72 | h |
| 40 | Sham | 4 | w |
| 41 | Sham | 5 | w |

TABLE 4
Probes derived from the CCl₄ model.

| | | | | Time after | | |
|---|---|---|---|---|---|---|
| Probe | Day of | Type of | Number of | last | | |
| number | experiment | treatment | treatments | treatment | | model |
| 9 | 2 | Olive oil | 1 | 1 | day | control |
| 10 | 3 | Olive oil | 1 | 2 | days | control |
| 11 | 6 | Olive oil | 2 | 3 | days | control |
| 12 | 9 | Olive oil | 3 | 3 | days | control |
| 13 | 12 | Olive oil | 4 | 3 | days | control |
| 14 | 15 | Olive oil | 3 | 9 | days | control |
| 15 | 15 | Olive oil | 5 | 3 | days | control |
| 16 | 16 | Olive oil | 6 | 1 | day | control |
| 17 | 17 | Olive oil | 6 | 2 | days | control |
| 18 | 18 | Olive oil | 6 | 3 | days | control |
| 19 | 21 | Olive oil | 6 | 6 | days | control |
| 20 | 24 | Olive oil | 6 | 9 | days | control |
| 49 | 36 | Olive oil | 12 | 3 | days | control |
| 50 | 39 | Olive oil | 12 | 6 | days | control |
| 51 | 42 | Olive oil | 12 | 9 | days | control |
| 52 | 45 | Olive oil | 12 | 12 | days | control |
| 53 | 48 | Olive oil | 12 | 15 | days | control |
| 21 | 1 | CCl₄ | 1 | 1 | day | Acute |
| 22 | 2 | CCl₄ | 1 | 2 | days | Acute |
| 23 | 3 | CCl₄ | 1 | 3 | days | Acute |
| 24 | 6 | CCl₄ | 2 | 3 | days | Acute |
| 25 | 9 | CCl₄ | 3 | 3 | days | Intermediate |
| 26 | 12 | CCl₄ | 3 | 6 | days | Recovery |
| 27 | 12 | CCl₄ | 4 | 3 | days | Intermediate |
| 28 | 15 | CCl₄ | 3 | 9 | days | Recovery |
| 29 | 15 | CCl₄ | 5 | 3 | days | Chronic |
| 30 | 16 | CCl₄ | 6 | 1 | day | Acute |
| 31 | 17 | CCl₄ | 6 | 2 | days | Acute |
| 32 | 18 | CCl₄ | 6 | 3 | days | Chronic |
| 33 | 21 | CCl₄ | 6 | 6 | days | Recovery |
| 34 | 24 | CCl₄ | 6 | 9 | days | Recovery |

Results of Hybridization Analysis

Standard quality control measurements and statistical analysis procedures were performed. The 6168 clones in the BDL model and the 4984 clones in the CC14 model were differentially expressed. Clones annotated as ESTs, putative/hypothetical proteins or unknown genes were subjected to advanced annotation. This included contig assembly (for ESTs), ORF and advanced homology searches and domain analysis.

Hierarchical clustering of the hybridizations showed the following main features:

BDL Model

The BDL model and sham (control) created distinct groups and within the BDL group the long term treatments (4 and 5 weeks) were close to one another and were separated from the short term treatments.

CCl₄ Model

Three main distinct groups were identified:

1) Most of the acute and chronic CCl₄ treatments;

2) Olive oil controls of treatments from group 1;

3) Recovery and their olive oil controls.

Within these groups it is interesting to note that the olive oil and CCl₄ treatments following 12 doses, created a subset of closely related groups. This rapid recovery demonstrates the reversibility of liver fibrosis in rat, even at a stage of micronodular cirrhosis, as was evident by fibrosis evaluation of liver samples.

PLD3 was identified to be upregulated (around 10 fold) by CCL₄ treatment and upregulated (around 2 fold) by BDL treatment.

### Example 5

### In Situ Hybridization Analysis of PDL3 Expression in Rat Liver Fibrosis

A fragment of the Phospholipase D3 cDNA (clone RLF-14F6) was used as the template for the synthesis of antisense (T3) and sense (T7) riboprobes. Radioactively labeled riboprobes were hybridized to the following rat liver samples:

Rat liver Multiblock 3 (RLMB3) representing a set of liver samples fixed at different time points after common bile duct ligation (BDL):

Sample: A—24 hr after sham operation
B—72 hr after sham operation
C—6 hr of BDL
E—24 hr of BDL
F—48 hr of BDL
G—72 hr of BDL
H—7 days of BDL
I—4 weeks of BDL
J—5 weeks of BDL

Rat liver multiblock 4 (RLMB4) represents a set of liver samples collected at different time points after CCl₄ poisoning:

Sample: K—untreated control liver sample
L—liver from vehicle (olive oil) injected animal
M—1 d after single CCL₄ treatment
N—3 d after single CCL₄ treatment
O—3 d after three CCL₄ treatments
P—9 d after three CCL₄ treatments
Q—3 d after five CCL₄ treatments
R—1 d after six CCL₄ treatments
S—3 d after six CCL₄ treatments
T—9 d after six CCL₄ treatments

Hybridization with sense (T7) probe resulted in no signal in all samples.

Pattern of the antisense (T3) hybridization signal is presented in tables 5 and 6 below:

TABLE 5
Pattern of the antisense (T3) hybridization signal against samples from Rat liver Multiblock 3 representing
a set of liver samples fixed at different time points after common bile duct ligation (BDL)

| | | | | | Vascular | | | | |
|---|---|---|---|---|---|---|---|---|---|
| Hepatocytes | | | | | elements | | | | |
| | | Necrotic/in | | Inflammatory | | In | Mesenchymal | | |
| Time | Norm. | areas | Sinusoidal | Infiltrating | | fibrotic | elements in | Cholangiocytes | |
| point | morph | ofnecrosis | Cells | cells | Normal | areas | fibrotic areas | Normal | Proliferating |
| 24 h sham | − | NP | ± | NP | − | NP | NP | − | NP |
| 72 h sham | − | NP | ± | + | − | NP | NP | − | NP |
| 6 h BDL | − | − | + | + | − | NP | NP | − | NP |
| 24 h BDL | − | − | + | ± | − | NP | NP | − | − |
| 48 h BDL | − | − | + | + | − | NP | − | − | − |
| 72 h BDL | − | − | + | + | − | NP | − | − | − |
| 7 d BDL | − | − | + | + | − | − | + | NP | − |
| 4 w BDL | − | − | ++ | ++ | − | − | ++ | NP | − |
| 5 w BDL | − | − | ++ | ++ | − | − | ++ | NP | − |

NP—not present;
− indicates no signal;
+ indicates positive signal;
++ indicates high signal

TABLE 6
Pattern of the antisense (T3) hybridization signal against samples from Rat liver Multiblock 4 which
represents a set of liver samples collected at different time points after CCl₄ poisoning:

| | | | | | Vascular | | | |
|---|---|---|---|---|---|---|---|---|
| Hepatocytes | | | | Inflamm. | elements | | Mesenchymal | |
| Time | Norm. | Necrotic/in areas | Sinusoidal | Infiltr. | | In | elements in | |
| point | morph | of necrosis | cells | cells | Normal | fibrotic areas | fibrotic areas | Cholangiocytes |
| Untreated control | − | NP | ± | NP | − | NP | NP | − |
| Vehicle control | − | NP | ± | NP | − | NP | NP | − |
| X1 + 1 d | − | − | + | − | − | NP | NP | − |
| X1 + 3 d | − | − | + | + | − | NP | ++ | − |
| X3 + 3 d | − | − | + | + | − | ++ | ++ | − |
| X3 + 9 d | − | − | ± | ± | − | − | − | − |
| X5 + 3 d | − | − | + | ++ | − | ++ | ++ | − |
| X6 + 1 d | − | − | + | ++ | − | + | ++ | − |
| X6 + 3 d | − | − | ± | ± | − | ++ | ++ | − |
| X6 + 9 d | − | − | ± | ± | − | ++ | ++ | − |

NP—not present;
− indicates no signal;
+ indicates positive signal;
++ indicates high signal

In control liver tissue (untreated, vehicle treated and sham operated), PLD3 gene was found to be expressed at low level in single sinusoidal cells located mainly at the periphery of hepatic lobules (periportal area). Sections of sample B (72 hr after sham operation) contained small fibrotic foci in subcapsular areas resulting most probably from foreign body reaction. Mesenchymal cells (showing positive αSMA staining on parallel sections) in this area showed strong hybridization signal as well as macrophages adjacent to foreign body.

Drastic changes in the intensity and pattern of expression occurred in pathological conditions especially after CCl₄ poisoning. Even a single poisoning resulted in accumulation of multiple expressing mesenchymal and inflammatory cells in the central part of the lobules. Increasing CCL₄ poisoning treatments are characterized by production of progressive fibrosis and parenchymal necrosis. In these pathological areas, there was also an increase in the number of mesenchymal and inflammatory cells expressing PLD3 gene. The expressing cells were located at the septal edges while fibrogenic cells within septa showed little or no hybridization signal. The pattern of the hybridization signal suggests that constitutive expression of PLD3 in quiescent stellate cells as well as upregulation of PLD3 expression in activated stellate cells are involved in fibrogenesis. Upon formation of fibrotic septa, myofibroblasts surrounded by collagen deposits appeared to down-regulate expression while myofibroblasts at the septal border maintained high level of expression. Another cell type relevant to fibrosis - inflammatory cells infiltrating the necrotic area also showed expression of the PLD3 gene. Significantly, mesenchymal cells and possibly endothelial cells involved in sinusoidal capillarization also showed a strong hybridization signal. Thus, this in situ hybridization study indicated the involvement of PLD3 in both fibrogenesis and cirrhotic tissue remodelling resulting from CCl₄ poisoning.

BDL samples showed slower up-regulation of PLD3 expression and increase in the number of expressing cells. At early time points (up to 72 hr of BDL) there was only slight up-regulation of expression in sinusoidal cells while expanding portal areas were free of hybridization signal. Later on (starting from 7 days of BDL) increasing number of PLD3 expressing cells accumulated at the periphery of fibrotic septa ingrowing into lobular parenchyma and in inflammatory cells.

Altogether, the results of the in situ hybridization study demonstrated a relevant expression pattern of the PLD3 gene, in both hepatic diseases models—CCL₄ and BDL, which indicate the involvement of this gene in hepatic inflammation, fibrogenesis and cirrhosis.

### Example 6

### In vitro Validation of PLD3 Function in Fibrosis

To evaluate the functional relationship of PLD3 to fibrosis, in vitro analysis of inhibition of its expression is performed. Cells expressing either the full-length gene or siRNA directed toward the PLD3 gene are established. Consequently, TGFβ stimulation is used as a fibrotic trigger and collagen accumulation, as well as the rate of proliferation and extra-cellular matrix state are monitored.

The amount of collagen is measured by Sirius Red dye reagent, and the optical density (OD) is measured at 530 nm against sodium hydroxide as a blank.

To determine cell number in each well, cells are detached by trypsinization and counted by means of a haemocytometer.

Cells overexpressing the full length gene are expected to be fibrotic while in cells expressing the siRNA the collagen content is expected to be reduced compared to normal cells exposed to TGFbeta treatment

### Example 7

### Examples of Models of Kidney Fibrosis

ZDF Rats

Samples of 9-month-old ZDF rats (Zucker diabetic fatty rats) presented hydronephrotic kidneys with dilated calyces. Microscopically these samples presented a picture of glomerulosclerosis and tubulointerstitial fibrosis. In accordance with these morphological changes, the expression of marker genes as measured by in situ hybridization (osteopontin (OPN), transforming growth factor β1 (TGF-β1) and procollagen α1(I) (Col1)) was significantly changed when compared to normal kidneys. Strong OPN expression was detectable in all tubular structures in both cortex and medulla. The TGF-β1 expression was widespread throughout interstitial cells. Some epithelial cells also showed TGF-β1 expression. Coll expression was detectable by in situ hybridization in most interstitial cells within the medulla, while cortical expression was “focal”.

Aged fa/fa (Obese Zucker) Rats

Samples of 12-month-old fa/fa rats presented strong glomerulosclerosis and diffuse tubulointerstitial fibrosis throughout the cortex and the medulla. The pattern of marker gene expression corresponded to morphological changes. OPN was expressed by tubular structures in the cortex and the medulla. Multiple interstitial cells expressed TGF-β1. Significantly, multiple foci and single interstitial cells showed strong Col1 expression in both cortex and medulla so that the number of Col1-expressing cells appeared to be higher in fa/fa samples than in ZDF samples.

Interestingly, Col1 expression was not detected in glomeruli of either ZDF or fa/fa rats in spite of the prominent accumulation of collagen, as revealed by Sirius Red staining. This suggested a low steady state level of Coll mRNA in glomerular cells.

Aged SD (Normal) Rats

Samples of aged SD rats showed increased accumulation of collagen in glomemli and interstitial space and increased expression of the marker genes. Significantly, the intensity of fibrotic change varied among samples so that one of four samples studied displayed very few changes compared with young animals; fibrotic change in another sample was confined to “polar” regions, and two samples showed uniform accumulation of collagen and elevated expression of marker genes throughout the sections.

Goto Kakizaki (GK)/Wistar (Normal) 48-Week-Old Rats

Samples of both GK and Wistar 48-week-old rats showed an accumulation of collagen in glomeruli and interstitial space. This accumulation was more pronounced in the GK samples. Two samples were used for mRNA isolation: C9 and GK9. Both were hybridized to the probe specific for IGFBP4. The in situ hybridization results showed that the GK sample demonstrated elevated expression of this gene.

Permanent UUO

Another known animal model in which mainly kidney fibrosis is evident, but without a background of diabetes, is unilateral ureteral obstruction (UUO) in which interstitial fibrosis is rapid and occurs within days following the obstruction.

A known model for fibrosis was employed—unilateral urether occlusion (UUO). One of the urethers was occluded (see below) and animals were sacrificed 1, 5, 10, 15, 20 and 25 days following occlusion.

Permanent UUO resulted in rapid activation (5 days of UUO) of collagen synthesis by interstitial cells in both medulla and cortex. By 20-25 days of UUO, significant amounts of interstitial collagen were deposited in the interstitial space while glomerular accumulation of collagen was confined to the outer capsule. Thus, permanent UUO samples provided an acute model of tubulointerstitial renal fibrosis without prominent glomerulosclerotic changes.

⅚ nephrectomy is another useful animal model for chronic renal insufficiency (CRI) in which fibrosis is evident.

The above models can be used as model systems for testing the therapeutic efficacy of the inhibitors of PLD described herein.

### Example 8

### Protocol for Permanent Unilateral Ureteral Obstruction (UUO)

Test System

Strain: Male Sprague-Dawley rats (9 weeks of age)

Group-Size: n=5 for operated rat, n=3 for sham-operated rats

Number of groups: 6 for both sham-operated and operated (i.e., 1 day, 5 days, 10 days, 15 days, 20 days and 25 days post-operation or post-sham operation)

Procedure

Rats were anaesthetized with Ketamin/Xylazine and the abdominal cavity was opened. After being exposed, the ureter from the right kidney was ligated with a suture over it (UUO). In sham-operated rats, the ureter was exposed but not ligated.

Study Termination

The study was terminated 24 hr, 5 days, 10 days, 15 days, 20 days and 25 days after the UUO procedure or after the sham operation. At this time point, the rats were sacrificed by exsanguination under CO₂ asphyxiation in order to collect the right kidney. After the capsule was removed the kidney was cut transversely. Half was fixed in 10% buffered formalin and the other half was immediately transferred to an eppendorf tube and frozen in liquid nitrogen for RNA analysis.

### Example 9

Expression of PLD3 in Rat UUO

Expression of PLD3 was evaluated following 7 and 14 days of obstruction of the ureter (UUO model) in rats and following 7 days obstruction in mice essentially as described in Example 8.

The expression of the gene was evaluated in kidney extracts using real-time PCR technology and was compared to the expression of control gene within these kidneys. For each time point kidneys from 5 mice or rats were used.

Increase in PLD3 expression was observed in all obstructed kidneys compared to sham operated mice or rats. The level of expression was increased 2-fold following 7 days obstruction in both mice and rats and reached 2.5-fold overexpression following 14 days in the rat UUO model.

Throughout this application various patent and scientific publications are cited. The disclosures for these publications in their entireties are hereby incorporated by reference into this application to more fully describe the state of the art to which this invention pertains.

1. A method of treating a fibrosis-related pathology in a subject which comprises administering to the subject a therapeutically effective amount of a pharmaceutical composition comprising a Phospholipase D3 inhibitor so as to thereby treat the subject.

2. The method of claim 1 wherein the pharmaceutical composition comprises an oligoribonucleotide or oligonucleotide which down-regulates the expression of gene Phospholipase D3 by at least 50% as compared to a control.

3. (canceled)

4. The method of claim 1 wherein the Phospholipase D3 inhibitor is an antisense oligonucleotide.

5. The method of claim 1 wherein the Phospholipase D3 inhibitor is a Phospholipase D3 siRNA.

6. The method of claim 1 wherein the Phospholipase D3 inhibitor is an expression vector comprising a nucleic acid molecule encoding Phospholipase D3 siRNA.

7. The method of claim 1 wherein the Phospholipase D3 inhibitor is an antibody which binds specifically to Phospholipase D3 polypeptide.

8. The method of claim 1 wherein the fibrosis-related pathology is chronic renal insufficiency, chronic renal insufficiency, nephropathy, liver fibrosis, cardiac fibrosis or kidney fibrosis.

9. The method of claim 1 wherein the fibrosis-related pathology is ocular scarring or cataract.

10-18. (canceled)

19. A pharmaceutical composition for the treatment of fibrosis comprising as an active ingredient a Phospholipase D3 inhibitor together with a pharmaceutically acceptable carrier.

20. (canceled)

21. The pharmaceutical composition of claim 19 wherein the Phospholipase D3 inhibitor is an oligoribonucleotide or oligonucleotide which down-regulates the expression of gene Phospholipase D3 by at least 50% as compared to a control.

22. The pharmaceutical composition of claim 19 wherein the Phospholipase D3 inhibitor is an antisense oligonucleotide.

23. The pharmaceutical composition of claim 19 wherein the Phospholipase D3 inhibitor is a Phospholipase D3 siRNA.

24. The pharmaceutical composition of claim 19 wherein the Phospholipase D3 inhibitor is an expression vector comprising a nucleic acid molecule encoding Phospholipase D3 siRNA.

25. The pharmaceutical composition of claim 19 wherein the Phospholipase D3 inhibitor is an antibody which binds specifically to Phospholipase D3 polypeptide.

26. The pharmaceutical composition of claim 19 wherein the fibrosis-related pathology is chronic renal insufficiency, chronic renal insufficiency, nephropathy, liver fibrosis, cardiac fibrosis or kidney fibrosis.

27. The pharmaceutical composition of claim 19 wherein the fibrosis-related pathology is ocular scarring or cataract.

28-30. (canceled)
